(19) United States
(12) Patent Application Publication
(10) Pub. No.: US 2016/0310591 A1
(43) Pub. Date: Oct. 27, 2016

(54) METHODS AND COMPOSITIONS FOR OVERCOMING DRUG-RESISTANCE IN CANCER BY TARGETED DELIVERY OF PRO-DRUG-NANO-POLYMERS

(71) Applicant: Northeastern University, Boston, MA (US)

(72) Inventors: Ban-An Khaw, Boston, MA (US); Prashant Bhattarai, Allston, MA (US)

(21) Appl. No.: 15/135,543

(22) Filed: Apr. 21, 2016

Related U.S. Application Data
(60) Provisional application No. 62/150,501, filed on Apr. 21, 2015.

(51) Int. Cl. A61K 39/395; A61K 9/00; C07K 16/44; A61K 31/337; A61K 31/198; C07K 16/40

(52) U.S. Cl. CPC A61K 39/3955; A61K 31/198; C07K 16/40; C07K 16/44; A61K 31/337; A61K 9/0019; C07K 2317/55; C07K 2317/31

(57) ABSTRACT

The present invention provides methods for targeted delivery of agents (e.g., drugs) to cells (e.g., cancer cells) using agent-polymer conjugates and bispecific targeting molecules. The invention further provides compositions and kits for practicing the targeted delivery methods.

### RELATED APPLICATION

This application claims the benefit of U.S. Provisional Application No. 62/150,501, filed on Apr. 21, 2015. The entire teachings of the above application are incorporated herein by reference.

### BACKGROUND

The development of cytotoxic agents that act on cancer cells, including various chemotherapeutic drugs, has resulted in significant progress in the field of cancer therapy, and the administration of such agents has been a focus of conventional therapeutic methods. However, as cancer progresses in a patient, cancer cells often acquire drug resistance through various mechanisms that allow the cells to evade drug-induced cell death. Such drug resistance can lead to the failure of chemotherapy. Hence, treating drug-resistant cancers is a significant challenge.

Recently, combination chemotherapy using multiple chemotherapeutic drugs has been shown to be effective in the treatment of certain cancers. The success of combination therapy has been mainly attributed to its ability to target different aspects of cancer cell physiology. However, combinations of chemotherapeutic agents can, in some cases, lead to drug antagonism, limiting the effectiveness of such combination therapies.

Accordingly, there is a need to develop methods and compositions for treating drug-resistant cancers more effectively, and for administering multiple therapeutic agents without inducing drug antagonism.

### SUMMARY OF THE INVENTION

Conventional non-targeted methods of delivering cytotoxic agents to cancer cells can be effective for certain cancers. However, many cancers, particularly cancers with drug-resistant cancer cells, do not respond well to non-targeted therapies. For such cancers, it is often desirable to employ targeted delivery of a therapeutic agent.

The present invention is based, in part, on the discovery that agent-polymer conjugates delivered to a cancer cell have certain advantageous properties that enhance targeted cancer therapy, particularly for drug-resistant cancers. The present invention is further based, in part, on the discovery that agent-polymer conjugates can be used to delivery multiple therapeutic agents in combination therapy approaches, without inducing significant drug antagonism.

Thus, in one embodiment, the invention provides a method for inhibiting the growth or metastasis of a cancer cell. The method generally comprises the step of contacting a cancer cell with a bispecific targeting molecule under conditions in which the bispecific targeting molecule binds to the cancer cell. The method further comprises the step of contacting a cancer cell that is bound to the bispecific targeting molecule with a plurality of agent-polymer conjugates under conditions in which the bispecific targeting molecule that is bound to the cancer cell also binds to a target moiety on at least one agent-polymer conjugate. In a particular embodiment, the plurality of agent-polymer conjugates includes multiple agent-polymer conjugates comprising at least two different agents for inhibiting the growth or metastasis of a cancer cell covalently linked to a polymeric carrier. In another embodiment, the plurality of agent-polymer conjugates comprises a mixture of different single-agent polymer conjugates. In yet another embodiment of the method, the plurality of agent-polymer conjugates comprises a combination of multiple agent-polymer conjugates and single agent-polymer conjugates.

The invention also provides, in additional embodiments, a method of treating a cancer in a subject in need thereof. The method generally comprises the steps of administering to the subject a bispecific targeting molecule and administering to the subject a plurality of agent-polymer conjugates. The agent-polymer conjugates administered to the subject comprise one or more agents that are delivered into cancer cells in the subject, thereby treating cancer in the subject. In a particular embodiment, the subject is a human. In a further embodiment, the subject is a human having a drug-resistant cancer.

The invention further provides, in other embodiments, compositions comprising a plurality of agent-polymer conjugates of the invention. In an embodiment, the plurality of agent-polymer conjugates comprise a population of multiple agent-polymer conjugates, each multiple agent-polymer conjugate comprising at least two different agents for inhibiting the growth or metastasis of a cancer cell covalently attached to a polymeric carrier. In another embodiment, the plurality of agent-polymer conjugates comprise a mixture of at least two different populations of single agent-polymer conjugates, each single-agent polymer conjugate comprising an agent for inhibiting the growth or metastasis of a cancer cell covalently linked to a polymeric carrier, wherein each population in the mixture comprises a different agent in comparison to other populations in the mixture.

The invention also provides, in further embodiments, a kit comprising a bispecific targeting molecule of the invention, agent-polymer conjugates of the invention, and a pharmaceutically acceptable carrier or excipient.

The methods and compositions described herein allow for effective targeted delivery of multiple agents (e.g., chemotherapeutic agents) to cancer cells and provide certain advantages, including the delivery of high concentrations of multiple agents to cancer cells without inducing drug antagonism. The methods and compositions of the invention are particularly useful for the treatment of drug-resistant cancers.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1A-1B.: Characterization of agent-polymer conjugate. 1A) Thin Layer Chromatography (TLC) to determine the conjugation of Paclitaxel to PGA. 1B) Anti-DTPA ELISA analysis carried out to determine the conjugation of DTPA to the polymer.

FIG. 2.: Binding specificity of bispecific biotinylated anti-DTPA to biotin receptors in various cell lines.

FIG. 3.: In vitro determination of cytotoxicity of agent-polymer conjugates incubated for 24 hours in SKOV-3 sensitive Ovarian cancer cells. The single agent-polymer conjugate Doxorubicin-DTPA-PGA (D-Dox-PGA), Paclitaxel-DTPA-PGA (D-PTXL-PGA) or Melphalan-DTPA-PGA (D-MEL-PGA) was incubated with SKOV-3 sensitive Ovarian cancer cells pre-targeted with bispecific anti-Her-2 Affibody-anti-DTPA antibody. For experiments with combination of agent-polymer conjugates, two or three of the single agent-polymer conjugate described above are incubated simultaneously in SKOV-3 sensitive Ovarian cancer cells pretargeted with bispecific anti-Her-2 Affibody-anti-DTPA antibody.

FIGS. 4A-4B.: In vitro determination of cytotoxicity of agent-polymer conjugates incubated for 24-48 hours in SKOV-3TR resistant Ovarian cancer cells. The free agent (DOX, PTXL or MEL) or single agent-polymer conjugate Doxorubicin-DTPA-PGA (D-Dox-PGA), Paclitaxel-DTPA-PGA (D-PTXL-PGA) or DTPA-Melphalan-PGA (D-MEL-PGA) was incubated in SKOV-3TR resistant Ovarian cancer cells pretargeted with 20 μg/ml of bispecific anti-Her-2 Affibody-anti-DTPA antibody. For experiments with combination of agent-polymer conjugates, two or three of the single agent-polymer conjugate described above are incubated simultaneously in SKOV-3TR resistant Ovarian cancer cells pretargeted with 20 μg/ml of bispecific anti-Her-2 Affibody-anti-DTPA antibody. 4A) cytotoxicity studies with 24 hours incubation of agent-polymer conjugates in SKOV-3TR resistant Ovarian cancer cells. 4B) cytotoxicity studies with 48 hours incubation of agent-polymer conjugates in SKOV-3TR resistant Ovarian cancer cells.

FIGS. 5A-5B.: In vitro determination of cytotoxicity of agent-polymer conjugates incubated for 48 hours in SKOV-3TR resistant Ovarian cancer cells. The free agent (DOX or PTXL) or single agent-polymer conjugate Doxorubicin-DTPA-PGA (D-Dox-PGA) or Paclitaxel-DTPA-PGA (D-PTXL-PGA) was incubated in SKOV-3TR resistant Ovarian cancer cells pretargeted with 40 μg/ml of bispecific biotinylated-anti-DTPA antibody. For experiments with combination of agent-polymer conjugates, two of the single agent-polymer conjugate described above are incubated simultaneously in SKOV-3TR resistant Ovarian cancer cells pretargeted with 40 μg/ml of bispecific biotinylated-anti-DTPA antibody. 5A) cytotoxicity studies represented as a plot of % cell viability plotted against Equivalent drug concentration in μg/ml. 5B) cytotoxicity studies represented as a bar chart of % cell viability vs Equivalent drug concentration in μg/ml.

FIG. 6.: In vitro determination of cytotoxicity of agent-polymer conjugates incubated for 48 hours in MCF-7 MDR doxorubicin resistant mammary carcinoma cells. The free agent (DOX or PTXL) or single agent-polymer conjugate Doxorubicin-DTPA-PGA (D-Dox-PGA) or Paclitaxel-DTPA-PGA (D-PTXL-PGA) was incubated in MCF-7 MDR cells pretargeted with 40 μg/ml of bispecific biotinylated-anti-DTPA antibody. For experiments with combination of agent-polymer conjugates, two of the single agent-polymer conjugate described above are incubated simultaneously in MCF-7 MDR cells pretargeted with 40 μg/ml of bispecific biotinylated-anti-DTPA antibody.

FIG. 7.: Comparison of IC50 values of Paclitaxel or Paclitaxel-DTPA-PGA (D-PTXL-PGA) in SKOV-3 sensitive and SKOV-3 TR resistant Ovarian cancer cells.

FIG. 8.: Epi-Fluorescent microscopy of MCF7-Doxorubicin resistant cells incubated with free Doxorubicin for 5 hours (panel A), free Doxorubicin for 1 hour followed by wash and incubation in fresh Doxorubicin free media (panel B). MCF7-Doxorubicin resistant cells were pretargeted with bispecific biotinylated-anti-DTPA antibody (sbAbCx) and incubated with D-Dox-PGA for 1 hour followed by wash and incubation in fresh Doxorubicin free media (panel C).

FIG. 9.: Cytotoxic effects of free agents and various agent-polymer conjugates studied in H9C2 rat cardiomyocytes.

### DETAILED DESCRIPTION OF THE INVENTION

### Methods for Inhibiting the Growth or Metastasis of Cancer Cells

The present invention, in certain embodiments, provides methods for inhibiting the growth or metastasis of cancer cells. The methods generally comprise the step of contacting a cancer cell with a bispecific targeting molecule under conditions in which the bispecific targeting molecule binds to the cancer cell. The methods further comprise the step of contacting a cancer cell that is bound to the bispecific targeting molecule with a plurality of agent-polymer conjugates under conditions in which the bispecific targeting molecule that is bound to the cancer cell also binds to a target moiety on at least one agent-polymer conjugate.

The term “inhibiting” as used herein, is understood to refer to reducing, decreasing, blocking or preventing.

The term “growth,” as used herein, refers to an increase in cell size and/or cell number (e.g., cell proliferation) as a result of cell growth and cell division processes. For example, cell growth can be the result of processes that are independent of normal cell-cycle regulatory mechanisms (e.g., loss of contact inhibition). In another instance, cell growth can result in uncontrolled cell division leading to the formation of new cells that have the ability to mutate and become a tumor.

The term “metastasis,” as used herein, refers to the physiological process by which cancer cells move from a primary location of a cancer to one or more other sites (e.g., in a subject). For example, metastasis can occur when cells break away from a cancerous tumor and travel through the bloodstream or through lymph vessels to other areas of a subject. Cancer cells that travel through the blood or lymph vessels can spread to other organs or tissues in distant parts of the subject.

As used herein, a “cancer cell” refers to both cancerous cells and pre-cancerous cells (e.g., cancer stem cells).

The methods for inhibiting the growth or metastasis of cancer cells described herein generally comprise the step of contacting a cancer cell with a bispecific targeting molecule under conditions in which the bispecific targeting molecule binds to the cancer cell. Conditions under which a bispecific targeting molecule binds to a cancer cell can be readily determined by a person of ordinary skill in the art, and include, for example, physiological conditions (e.g., when the cancer cell is present in a subject).

As used herein, a “bispecific targeting molecule” or “bispecific targeting ligand” refers to a molecule that comprises at least two specific binding sites for binding at least two distinct molecules, wherein the bispecific targeting molecule can specifically bind both molecules simultaneously. A person of skill in the art would understood that a bispecific targeting molecule can include more than two binding sites (e.g., 3, 4, 5 binding sites, etc.), provided the targeting molecule includes at least one binding site for each of two targets. In certain embodiments, the bispecific targeting molecule includes only two binding sites. In general, bispecific targeting molecules act as targeting agents, bringing other molecules to the site of interest.

Bispecific targeting molecules can include, but are not limited to, formats such as “Bispecific Antibody-Antibody”; “Bispecific Antibody-Ligand”; “Bispecific Ligand-Ligand”; “Bispecific Affibody-Antibody” or a “Bispecific Affibody-Affibody”. In certain embodiments, the binding sites are joined to each other in specific relative orientations (e.g., joined with a regiospecific linkage).

Suitable methods of making and characterizing a bispecific targeting molecule are well known to a person skilled in the art and include, for example, methods exemplified herein (see, e.g., Examples 1 and 3).

In certain embodiments, the bispecific targeting molecules comprise an antibody, an antigen-binding fragment or a combination thereof. The term “antibody” is understood to refer to immunoglobulin molecules of any isotype, e.g., IgG, IgM, IgA1, IgA2, IgD, or IgE. The term “antigen-binding fragments” include, but are not limited to, a Fab fragment, a F(ab′)2 fragment, a Fd fragment, a Fv fragment, a dAb fragment, single chain Fv, a dimerized variable region (V region) fragment (diabody), a disulfide-stabilized V region fragment (dsFv), an affibody, an antibody mimetic, and one or more isolated complementarity determining regions (CDR) that retain specific binding to their cognate antigen.

In a particular embodiment, the bispecific targeting molecule comprises an anti-Her-2 Affibody and an anti-DTPA antibody. In an embodiment, the bispecific targeting molecule comprises a biotinylated-anti-DTPA antibody (sbAbCx).

The bispecific targeting molecules employed in the methods described herein include two or more (e.g., 2, 3, 4, 5, etc.) binding sites for two or more distinct molecules. Typically, the bispecific targeting molecules comprise at least one first binding site for a target antigen on the surface of the cancer cell and at least one second binding site for a target moiety on an agent-polymer conjugate molecule. The term “target antigen” as used herein, refers to any molecule that is present on the surface of a cancer cell that can be specifically bound by a binding site on a bispecific targeting molecule of the invention. The target antigen on the surface of the cancer cell that is recognized by the bispecific targeting molecule can be any cell surface-antigen, including, but not limited to, receptors (e.g., cell surface receptors, transmembrane receptors having an extracellular domain) and receptor ligands (e.g., ligands bound to receptors on the surface of a cancer cell).

To provide a binding site for a target antigen or moiety on a bispecific targeting molecule, the bispecific targeting molecule can include, for example, an antibody, antibody fragment, antibody mimetic, nucleic acid (e.g., aptamer), hapten (e.g., biotin), a molecule having affinity for a hapten (e.g., streptavidin, avidin, neutravidin), a biological protein (e.g., hormone, cytokine, receptor ligand), and carbohydrate. In certain embodiments, the binding site specifically binds to a molecule that is present in the sample or subject to which the target molecule is to be delivered. In certain embodiments, the binding site does not specifically bind to a molecule that is present in the sample or subject to which the target molecule is to be delivered. In certain embodiments, the binding site specifically binds to the target molecule. In certain embodiments, the binding site does not specifically bind to the target molecule.

“Specific” and “specificity” is used herein to refer to a selective interaction between two members of a specific binding pair (e.g., a ligand and a binding site, an antibody and an antigen). The phrase “specifically binds to” and analogous phrases refer to the ability of molecules in the binding pair to bind specifically to one another (e.g., without appreciable binding to other molecules).

Generally, the binding of a first binding site on a bispecific targeting molecule to a target antigen on the surface of the cancer cell does not sterically hinder the binding of a second binding site to a target moiety. In certain embodiments, the binding of at least one first binding site to a target antigen on the surface of the cancer cell occurs simultaneously with the binding of at least one second binding site to a target moiety. In other embodiments, the binding of at least one first binding site to a target antigen on the surface of the cancer cell and the binding of at least one second binding site to a target moiety occurs sequentially (e.g., the binding of at least one first binding site to a target antigen on the surface of the cancer cell occurs before the binding of at least one second binding site to a target moiety; the binding of at least one first binding site to a target antigen on the surface of the cancer cell occurs after the binding of at least one second binding site to a target moiety).

In some embodiments, the binding of at least one first binding site to a target antigen on the surface of the cancer cell and the binding of at least one second binding site to a target moiety (e.g., on an agent-polymer conjugate), occurs under the same set of conditions (e.g., pH, temperature, buffer composition), such as physiological conditions (e.g., in a subject). In other embodiments, the binding of at least one first binding site to a target antigen on the surface of the cancer cell and the binding of at least one second binding site to a target moiety occurs under different conditions (e.g., different pH conditions).

In accordance with the present invention, the method for inhibiting the growth or metastasis of a cancer cell further comprises the step of contacting a cancer cell that is bound to a bispecific targeting molecule with a plurality of agent-polymer conjugates.

An “agent-polymer conjugate” as used herein is a composition comprising at least one agent covalently attached to a polymeric carrier.

The term “agent” as used herein refers to any molecule or compound that is useful in the detection, diagnosis or treatment of a disease or disorder (e.g., cancer). The agent can be organic or inorganic, natural or synthetic, labeled or unlabeled (e.g., radioactive or non-radioactive). Examples of agents include, without limitation, chemotherapeutic agents (e.g., cell-cycle inhibitors, agents causing cell death, drugs, pro-drugs, microtubule inhibitors, DNA-cross linking agents, DNA-alkylators, PARP inhibitors, cMet inhibitors), radioisotopes, cytokines, pro-apoptotic agents and immune-activating agents. In certain embodiments, the agent is a therapeutic agent. In certain embodiments, the therapeutic agent is selected from the group consisting of doxorubicin (DOX), carbozantinib, 6-mercaptopurine, 6-thioguanine, cytarabine, 5-fluorouracil decarbazine, mechlorethamine, thioepa chlorambucil, CC-1065, Melphalan (MEL), carmustine (BSNU), lomustine (CCNU), cyclothosphamide, busulfan, dibromomannitol, streptozotocin, mitomycin, cis-dichlorodiamine platinum (II) (DDP) cisplatin, daunorubicin, dactinomycin, bleomycin, mithramycin, anthramycin (AMC), vincristine, vinblastine, taxol, Paclitaxel (PTXL), maytansinoids, cytochalasin B, gramicidin D, ethidium bromide, emetine, etoposide, tenoposide, colchicin, dihydroxy anthracin dione, mitoxantrone, mithramycin, 1-dehydrotestosterone, glucocorticoids, procaine, tetracaine, lidocaine, propranolol, puromycin, and calicheamicin.

The phrase “polymeric carrier” is understood to refer to any polymer to which one or more agents can be chemically/covalently linked. The polymers described herein comprise at least 3 monomers wherein each of the monomer is either an organic or inorganic molecule or a combination thereof. Organic molecules are usually composed of carbon atoms in rings or long chains, to which are attached other atoms of such elements as hydrogen, oxygen, and nitrogen. The polymeric carrier can be charged or uncharged. In certain embodiments, the polymeric carrier is negatively charged at a pH range of about 6.0-10.0. In a particular embodiment, the polymeric carrier is negatively charged at a physiological pH. The polymeric carrier can be hydrophilic, hydrophobic or amphipathic. The polymeric carrier can be branched or unbranched. The polymeric carrier can be peptidic, non-peptidic or a combination thereof. The term “peptidic” as used herein, refers to polymeric carriers having two or more amino acids linked in a chain, the carboxyl group of each acid being joined to the amino group of the next by a bond of the type —OC—NH—. The polymeric carrier may or may not elicit an immune response by itself.

In certain embodiments, the polymeric carrier is linear (i.e., unbranched, has only two ends). In certain embodiments, the polymeric carrier is branched (i.e., has more than two ends). In a certain embodiment, the polymeric carrier is negatively charged. In certain embodiment, the polymeric carrier is present in a molecule that consists essentially of the polymeric carrier, at least two payload molecules, and a target moiety. In certain embodiment, the polymeric carrier further comprises a spacer. In a certain embodiment, the polymeric carrier is covalently linked to DTPA on one of its terminal ends. In a certain embodiments, the polymeric carrier is covalently linked to DTPA on all of its terminal ends. In a certain embodiment, the polymeric carrier is covalently linked to at least one DTPA molecule. In a certain embodiment, the polymeric carrier is covalently linked to at least two DTPA molecules. In certain embodiment, the polymeric carrier is not linked to DTPA. In a certain embodiment, the polymeric carrier is homogenously modified to alter the properties of the polymeric carrier, e.g., decrease positive charge/increase negatively charge of the polymer, modify the solubility of the polymer, blocking reactive sites on the polymeric carrier. In a certain embodiment, the groups used for modification of the general properties of polymeric carrier are not agent molecules.

The polymeric carrier may be a homopolymer (e.g., made up of repeat units of the same monomer) or a heteropolymer (e.g., made up of different repeats units). Hydrophilic and hydrophobic monomers can be used as the monomers to in a heteropolymer. In certain embodiment, the polymeric carrier is selected from the group consisting of polylysine, polyglutamic acid (PGA), N-(2-hydroxypropyflmethacrylamide, polycation polymers, poly(allylamine), poly(dimethyldiallyammonim chloride) polylysine, poly(ethylenimine), poly(allylamine), natural polycations, dextran amine, polyarginine, chitosan, gelatine A, protamine sulfate, polyanion polymers, poly(styrenesulfonate), polyglutamic or alginic acids, poly(acrylic acid), poly(aspartic acid), poly(glutaric acid), natural polyelectrolytes with similar ionized groups, dextran sulfate, carboxymethyl cellulose, hyaluronic acid, sodium alginate, gelatine B, chondroitin sulfate, and heparin. In certain embodiments, polymeric carrier comprises monomers that are glucosamine, glucose and other amino-sugars (e.g., fructoseamine, galactosamine).

The polymeric carrier typically has a molecular weight of 0.5 kDa, 1 kDa, 2 kDa, 3 kDa, 5 kDa, 10 kDa, 15 kDa, 20 kDa, 25 kDa, 30 kDa, 35 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 250 kDa, 300 kDa, 350 kDa, 400 kDa, 450 kDa, 500 kDa, 600 kDa, 700 kDa, 800 kDa, 900 kDa, 1000 kDa or more. In certain embodiments, the polymeric carrier comprises peptide monomers linked by a plurality of peptide bonds. In one embodiment, the polymeric carrier comprises at least three peptide monomers. In one embodiment, the polymeric carrier comprises at least three identical peptide monomers. In one embodiment, the polymeric carrier comprises at least three different peptide monomers. In one embodiment, the polymeric carrier comprises between 3 to 200 peptide monomers. In one embodiment, the polymeric carrier comprises between 3 to 200 identical peptide monomers. In a particular embodiment, the polymeric carrier comprises between 3 to 200 glutamic acid monomers linked by a plurality of peptide bonds to form a poly glutamic acid polymeric carrier. In a different embodiment, the polymeric carrier comprises between 3 to 200 lysine monomers linked by a plurality of peptide bonds to form a poly lysine acid polymeric carrier.

In certain embodiments, the polymeric carrier comprises a structure set forth in formulae I or II:

(X)—Pₙ—(X), (I)

(X)—Pₙ—(Y), (II)

wherein (X), P and (Y) are independently an amino acid with a non-polar side chain, an amino acid with a polar side chain that is not charged at physiological pH, or an amino acid with a polar side chain that is charged at physiological pH; and wherein n is at least one (e.g., 1, 2, 3 or more).

The expression “non-polar side chain” as used herein, refers to a side chain “R” group of a naturally occurring or unnatural amino acid that is uncharged at physiological pH and cannot form or participate in a hydrogen bond. Examples of amino acid with non-polar side chain include, but not limited to, glycine (Gly), alanine (Ala), valine (Val), leucine (Leu), isoleucine (Ile), proline (Pro), phenylalanine (Phe), methionine (Met), and norleucine (Nle). Tryptophan (Trp) is a non-polar amino acid that is an exception due the presence of a hydrogen donor atom in its side chain. An amino acid with “non-polar side chain” is commonly known to those of skill in the art. The expression “polar side chain that is not charged at neutral pH” as used herein, refers to a side chain “R” group of a naturally occurring or unnatural amino acid that is substantially uncharged at physiological pH and has hydrogen donor or acceptor atoms in its side chain that can participate in a hydrogen bond. Examples of amino acid with polar side chain that is substantially uncharged at neutral pH include, but not limited to, serine (Ser), threonine (Thr), cysteine (Cys), asparagine (Asn), glutamine (Gln), and tyrosine (Tyr). An amino acid with “polar side chain that is not charged at neutral pH” is commonly known to those of skill in the art. The expression “polar side chain that is charged at neutral pH” as used herein, refers to a side chain “R” group of a naturally or unnaturally occurring amino acid that is either substantially charged at physiological pH or can participate in hydrogen bonding as it has hydrogen donor or acceptor atoms in its side chain. Examples of amino acid with polar side chain that is substantially charged at physiological pH include, but not limited to, arginine (Arg), lysine (Lys), ornithine (Orn) and histidine (His), aspartic acid or aspartate (Asp) and glutamic acid or glutamate (Glu). An amino acid with “polar side chain that is charged at neutral pH” is commonly known to those of skill in the art. The term “substantially” as used herein means “for the most part” or “predominantly” or “at least partially”. For example, glutamic acid is considered to be negatively charged at neutral pH as the carboxylic side chain loses an H+ ion (proton). In reality there exists an equilibrium between the negatively charged un-protonated form and the uncharged protonated form of glutamic acid in a peptide. Glutamic acid is considered to have a “substantial” negative charge at neutral pH because the equilibrium is shifted towards the un-protonated form and the “predominant” species in solution is the negatively charged species. The term “unnatural amino acid” or the phrase “unnaturally occurring amino acid” refers to any amino acid, modified amino acid, and/or amino acid analogue that is not one of the 20 naturally occurring amino acids or seleno cysteine. For example unnatural amino acids include, but are not limited to, D-enantiomers of 20 naturally occurring amino acids, ornithine and beta-lysine. Physiological pH refers to a pH value that normally prevails in the human body (e.g., a pH value of about 7.4). Neutral pH refers to a pH value of about 7.0.

In other embodiments, (X), P and (Y) forth in formulae I or II are molecules other than amino acids. For example, (X), P and (Y) can be independently glucose or an amino-sugar (e.g., glucosamine, fructoseamine, galactosamine).

“Covalently” or “covalent” as used herein is understood as a chemical bond between two atoms in which electrons are shared between them. Examples include, but not limited to, peptide bonds, disulfide bonds and non-natural chemical linkages. As used herein, “linked”, “linkage”, “joined” and the like refer to a juxtaposition wherein the components described are attached to each other in a relationship permitting them to function in their intended manner. The components can be linked covalently (e.g., peptide bond, disulfide bond, non-natural chemical linkage), through hydrogen bonding (e.g., knob-into-holes pairing of proteins, see, e.g., U.S. Pat. No. 5,582,996; Watson-Crick nucleotide pairing), or ionic binding (e.g., chelator and metal) either directly or through spacers (e.g., peptide sequences, typically short peptide sequences; nucleic acid sequences; or chemical linkers). In certain embodiments of the invention, spacers can be used to provide separation between the target moiety and the polymeric carrier so that the agent-polymer conjugate can bind without any steric hindrance to the bispecific targeting molecule. Spacers can also be used, for example, in joining binding sites to each other and/or joining agent molecules to polymeric carriers. In certain embodiments, spacers can be used to provide separation between agent molecules so that the activity of the agent molecules is not substantially inhibited (less than 10%, less than 20%, less than 30%, less than 40%, less than 50%) relative to the agent molecules directly linked to the polymeric carrier, under conditions in which the reagents of the invention are used, i.e., typically physiological conditions. In certain embodiments of the methods of the invention, the covalent linkage is a peptide linkage, an amide linkage, a sulfyhydrl linkage, a maleimide linkage, a thioester linkage, an ether linkage, an ester linkage, a hydrazine linkage, a hydrazine linkage, an oxime linkage or any other covalent linkages known to a person of skill in the art.

In certain embodiments, the agent-polymer conjugate comprises one or more agents that are covalently linked to the polymeric carrier in a prodrug form. As used herein, the term “prodrug” means a derivative of a compound that can hydrolyze, oxidize, metabolize or otherwise react under biological/physiological conditions (in vitro or in vivo) to provide the compound that can either inhibit/kill a cancer cell or inhibit different aspects of cancer cell physiology (e.g., growth, replication, proliferation and metastasis). Generally, a prodrug is a compound that, after administration, is metabolized (i.e., converted within the body) into a pharmacologically active drug. In some instances, prodrugs are pharmacologically inactive in systemic circulation and are converted into an active form within the body at a particular or specific site (e.g., cancer cell). In some instances, prodrugs are pharmacologically inactive before administration but are converted into an active form in the systemic circulation within the subject. In target cancer therapy, a prodrug is used reduce adverse effects of a drug due to non-targeted toxicities. In some embodiments, the agent-polymer conjugates comprises one or more doxorubicin molecules or paclitaxel molecules or melphalan molecules covalently linked to one or more polyglutamic acid (PGA) polymers in a prodrug form. In other embodiment, the agent-polymer conjugate comprises a combination of one or more of each doxorubicin and paclitaxel molecules covalently linked to a PGA polymer in a prodrug form. In yet another embodiment, the agent-polymer conjugate comprises a combination of one or more of each doxorubicin, paclitaxel and melphalan molecules covalently linked to a PGA polymer in a prodrug form.

In one embodiment, the plurality of agent-polymer conjugates comprises a population of multiple agent-polymer conjugates. The term “population” as used herein is understood to mean a group of two or more molecules having the same or substantially similar identity. The phrase “multiple agent-polymer conjugate” refers to a molecule comprising two or more distinct agents covalently attached to the same polymeric carrier. The multiple agent-polymer conjugate can include one or more (e.g., 2, 3, 4, 5, etc.) of each distinct agent present in the conjugate. In one embodiment, the multiple agent-polymer conjugate comprises at least two distinct therapeutic agents for inhibiting the growth or metastasis of a cancer cell. In another embodiment, the multiple agent-polymer conjugate comprises at least two distinct non-therapeutic agents. In yet another embodiment, the multiple agent-polymer conjugate comprises at least two agents, wherein at least one of the agents is a therapeutic agent and at least one agent is a non-therapeutic agent. Typically, the two or more distinct agents are linked to the polymeric carrier such that they do not sterically hinder or disrupt the specific interaction of the multiple agent-polymer conjugate with a bispecific targeting molecule.

In some embodiments, the plurality of agent-polymer conjugates comprises a mixture of at least two (e.g., 2, 3, 4, 5, etc.) different populations of single agent-polymer conjugates. The phrase “single agent-polymer conjugate” as used herein refers to a composition comprising only one type of agent covalently attached to a polymeric carrier. The term “type” as used herein refers to the physical (e.g., solubility) and chemical (e.g., chemical formula) properties of a molecule, agent or moiety. A single agent-polymer conjugate can include one or more (e.g., 2, 3, 4, 5, etc.) of the agent that is present in the conjugate. Typically, the agent is linked to the polymeric carrier such that it does not sterically hinder or disrupt the specific interaction of the multiple agent-polymer conjugate with a bispecific targeting molecule.

In accordance with the present invention, the method for inhibiting the growth or metastasis of a cancer cell further comprises the step of contacting a cancer cell that is bound to a bispecific targeting molecule with a plurality of agent-polymer conjugates, under conditions in which the bispecific targeting molecule that is bound to the cancer cell also binds to a target moiety covalently linked to at least one agent-polymer conjugate. Conditions under which a bispecific targeting molecule (e.g., that is bound to a cancer cell) binds to a target moiety on an agent-polymer conjugate can be readily determined by a person of ordinary skill in the art, and include, for example, physiological conditions (e.g., when the cancer cell is present in a subject).

As used herein, a “target moiety” means any chemical entity (e.g., molecule, functional group) that can be specifically bound by at least one binding site of a bispecific targeting molecule described herein. The bispecific targeting molecule can bind to 1, 2, 3, 4, 5, 6, 7, 8 or more target moieties on an agent-polymer conjugate. The target moiety typically has a molecular weight of about 10 kDa, 7 kDa, 5 kDa, 3 kDa, 2 kDa, 1 kDa, 750 Da, 500 Da or less. Suitable target moieties for inclusion in the agent-polymer conjugates described herein include, but are not limited to, DiethyleneTriaminePentaacetic Acid (DTPA), aniline and its carboxyl derivatives (o-, m-, and p-aminobenzoic acid); fluorescein, biotin, digoxigenin, and dinitrophenol. In general, the target moieties will not be naturally-occurring in subject being treated.

In a particular embodiment, the target moiety is present in one population of agent-polymer conjugates. In another embodiment, the target moiety is present in two or more different populations of agent-polymer conjugates.

In one embodiment, the method for inhibiting the growth or metastasis of a cancer cell comprises contacting a cancer cell with a bispecific anti-Her-2 Affibody-anti-DTPA antibody and a plurality of agent-polymer conjugates comprising a population of DTPA-Doxorubicin-Paclitaxel-Poly Glutamic acid (D-Dox-PTXL-PGA) conjugates, or a mixed population of DTPA-Doxorubicin-Poly Glutamic acid (D-Dox-PGA) conjugates and DTPA-Paclitaxel-Poly Glutamic acid (D-PTXL-PGA) conjugates (as shown in Examples 8-14).

### Methods for the Treatment of Cancer

The present invention also provides, in various embodiments, methods for treating cancer in a subject (e.g., a subject in need thereof). Generally, the cancer treatment method comprises administering to the subject a bispecific targeting molecule described herein and a composition comprising a plurality of agent-polymer conjugates of the invention.

As used herein, the terms “treat,” “treating,” or “treatment,” mean to counteract a medical condition (e.g., cancer) to the extent that the medical condition is improved according to a clinically-acceptable standard (e.g., inhibition of growth/metastasis of cancer cells, remission of a cancer, or cure of a cancer).

As used herein, “subject” refers to a mammal (e.g., human, non-human primate, cow, sheep, goat, horse, dog, cat, rabbit, guinea pig, rat, mouse). In a particular embodiment, the subject is a human. A “subject in need thereof” refers to a subject (e.g., patient) who has, or is at risk for developing, a disease (e.g., cancer) or condition that can be treated (e.g., improved, ameliorated, prevented) according to the methods described herein.

Cancers that can be treated using the methods described herein include, for example, hematological cancers and solid tumor cancers. Examples of solid cancers include breast cancer, ovarian cancer, colorectal cancer, pancreatic cancer, lung cancer, liver cancer, brain cancer, kidney cancer, prostate cancer, gastrointestinal cancer, melanoma, cervical cancer, bladder cancer, glioblastoma, melanoma, and head and neck cancer. Examples of hematological cancers include leukemias (e.g., acute myeloid leukemia (AML), acute monocytic leukemia, promyelocytic leukemia, eosinophilic leukemia, acute lymphoblastic leukemia (ALL) such as acute B lymphoblastic leukemia (B-ALL), chronic myelogenous leukemia (CML), chronic lymphocytic leukemia (CLL)), lymphomas (e.g., non-Hodgkin lymphoma, Hodgkin lymphoma), and myelodysplastic syndrome (MDS). In certain embodiments, the cancer is an ovarian cancer. In certain embodiments, the cancer is a lung cancer. In certain embodiments, the cancer is a breast cancer. In a particular embodiment, the cancer is a triple negative breast cancer.

In a particular embodiment, an effective amount of a composition comprising a plurality of agent-polymer conjugates is administered to a subject in need thereof. As defined herein, an “effective amount” refers to an amount of a bispecific targeting molecule and/or a composition comprising agent-polymer conjugates that, when administered to a subject, is sufficient to perform its intended function (e.g., detection, diagnosis or treatment of a cancer). A “therapeutically effective amount” refers to an amount of a bispecific targeting molecule and/or a composition comprising agent-polymer conjugates that, when administered to a subject, is sufficient to achieve a desired therapeutic effect in the subject under the conditions of administration, such as an amount sufficient to inhibit (e.g., prevent, reduce, eliminate) the growth/metastasis of cancer cells (e.g., drug resistant ovarian cancer cell) in the subject.

A person of skill in the art (e.g., a physician) will appreciate that certain factors may influence the effective (e.g., therapeutically effective) amount required to effectively treat a subject, including but not limited to the severity of cancer, previous treatments (e.g., sensitive or resistant to certain drugs), the general health and/or age of the subject, and other diseases present. Moreover, treatment of a subject with a therapeutically effective amount of a bispecific targeting molecule and/or a composition comprising plurality of agent-polymer conjugates can include a single treatment or a series of treatments. In one example, a subject is treated with a bispecific targeting molecule and a composition comprising plurality of agent-polymer conjugates once per week for between about 1 to 10 weeks, alternatively between 2 to 8 weeks, between about 3 to 7 weeks, or for about 4, 5, or 6 weeks. It will also be appreciated that the effective amount may increase or decrease over the course of a particular treatment regimen.

In some embodiments, an effective amount, or therapeutically effective amount of a bispecific targeting molecule and/or a composition comprising agent-polymer conjugates ranges from about 0.001 mg/kg body weight of the subject to about 100 mg/kg body weight of the subject, e.g., from about 0.01 mg/kg body weight to about 50 mg/kg body weight, from about 0.025 mg/kg body weight to about 25 mg/kg body weight, from about 0.1 mg/kg body weight to about 20 mg/kg body weight, from about 0.25 mg/kg body weight to about 20 mg/kg body weight, from about 0.5 mg/kg body weight to about 20 mg/kg body weight, from about 0.5 mg/kg body weight to about 10 mg/kg body weight, from about 1 mg/kg body weight to about 10 mg/kg body weight, or about 5 mg/kg body weight. In some other instances, a therapeutically effective amount of a bispecific targeting molecule and a composition comprising plurality of agent-polymer conjugates collectively range from about 0.001 mg/kg body weight of the subject to about 500 mg/kg body weight of the subject. In some instances, the effective amount or concentration of the agent in the composition comprising plurality of agent-polymer conjugates can range from about 0.001 mg to about 50 mg total, e.g., from about 0.01 mg to about 40 mg total, from about 0.025 mg to about 30 mg total, from about 0.05 mg to about 20 mg total, from about 0.1 mg to about 10 mg total, or from about 1 mg to about 10 mg total.

In various embodiments, the agents described herein are conjugated to a polymeric carrier in a prodrug form. The agent in the prodrug form is non-toxic, or exhibits reduced toxicity at the effective dose, when conjugated to the polymeric carrier. Without being bound by theory, it is believed that upon binding of an agent-polymer conjugate to a pretargeted bispecific targeting molecule (which is itself specifically bound to a target cancer cell), the agent-polymer conjugate is internalized by the cell. This mode of delivery ensures that the non-targeted toxicities resulting from the unintended, uncontrolled release of the agent in the agent-polymer conjugate is minimized. Thus, using the methods described herein, cancer cells can be targeted with increased safety.

In one embodiment, the agent in the prodrug form is only metabolized into an active drug inside a cancer cell. In a particular embodiment, the active drug is released into the cytoplasm of the cancer cell. In another embodiment, the active drug is released into the lysosome of the cancer cell. In certain embodiments, the active drug is not released into the systemic circulation of the subject. In certain embodiments, the active drug is released in the systemic circulation of the subject but does not cause toxicities associated with the corresponding free drug. In certain embodiments, the concentration of the active drug released into the cancer cell is higher than the maximum tolerated dose (MTD) of the corresponding free drug that is delivered to the cancer cell in an unconjugated form. In some instances, at least 0.5-fold, 1 fold, 2-fold 3-fold, 4-fold, 5-fold, 7-fold, 10-fold, 12-fold, 15-fold, 20-fold, more drug is delivered into the cancer cell using the method described herein than the corresponding free drug delivered by diffusion into a cancer cell. In certain embodiments, the concentration of the active drug released into the cancer cell is lower than the maximum tolerated dose (MTD) of the corresponding free drug that is delivered to the cancer cell in an unconjugated form. In one embodiment, the agent can be administered in a metronomic dosing regimen, whereby a lower dose is administered more frequently relative to maximum tolerated dosing. A number of preclinical studies have demonstrated superior anti-tumor efficacy, potent antiangiogenic effects, and reduced toxicity and side effects (e.g., myelosuppression) of metronomic regimes compared to maximum tolerated dose (MTD) counterparts (Bocci, et al., Cancer Res, 62:6938-6943, (2002); Bocci, et al., Proc. Natl. Acad. Sci., 100(22): 12917-12922, 4561.1001-000-9-2310246.v1(2003); and Bertolini, et al., Cancer Res, 63(15):4342-4346, (2003. In some instances, at least 0.5-fold, 1 fold, 2-fold 3-fold, 4-fold, 5-fold, 7-fold, 10-fold, 12-fold, 15-fold, 20-fold, less drug is delivered into the cancer cell using the method described herein than the corresponding free drug delivered by diffusion into a cancer cell.

In the method of treating a cancer, a bispecific targeting molecule is generally administered prior to the administration of a composition comprising plurality of agent-polymer conjugates. For example, a bispecific targeting molecule can be administered 4 hrs, 8 hrs, 12 hrs, 16 hrs, 20 hrs , 24 hrs, 36 hrs, 48 hrs, 72 hrs, 4 days, 5 days, 6 days, 7 days, or more prior to administration of a composition comprising plurality of agent-polymer conjugates.

In other embodiments, a bispecific targeting molecule is administered after the administration of a composition comprising plurality of agent-polymer conjugates. For example, the composition comprising plurality of agent-polymer conjugates can be administered first and bispecific targeting molecule is subsequently administered about 5 min later, 10 mins later, 15 mins later, 20 mins later, 25 mins later, 30 mins later, 35 mins later, 40 mins later, 45 mins later 50 mins later, 55 mins later, or 1 hr, 2 hrs, 3 hrs, 4 hrs, or more hours, later. In certain other instances, a bispecific targeting molecule and a composition comprising plurality of agent-polymer conjugates described herein are administered simultaneously.

The composition comprising plurality of agent-polymer conjugates can be administered to the subject as a prophylactic or therapeutic composition (e.g., to prevent or treat a disease or condition) or, alternatively, as a non-therapeutic composition (e.g., a diagnostic or labelling composition). The composition comprising plurality of agent-polymer conjugates can be administered to the subject to treat pre-existing dis-orders (e.g, drug resistant cancers). In addition to treating pre-existing disorders, the methods described herein can prevent or slow the onset/ metastasis of such disorders. For example, the bispecific targeting molecule and a composition comprising plurality of agent-polymer conjugates can be administered for prophylactic applications, e.g., can be administered to a subject susceptible to or otherwise at risk of developing cancer. In some instances, the bispecific targeting molecule and a composition comprising plurality of agent-polymer conjugates can be administered to a subject who has cancer stem cells or cells that have the potential to mutate into a cancer cell. The composition comprising plurality of agent-polymer conjugates can be administered to the subject to treat drug resistant cancers in a subject (e.g, relapsed subject).

The terms “administer”, “administering”, “administration” or any grammatical equivalent thereof include any method of delivery of a bispecific targeting molecule and/or composition comprising agent-polymer conjugates into a subject (e.g., to a particular region in or on a subject). The agent can be administered intravenously, intramuscularly, subcutaneously, intrathecally, intracereberal, intraventricular, intraspinal, intradermally, intranasally, orally, transcutaneously, or mucosally. In certain embodiments, the agent is administered by injection (e.g., intratumoral injection). A skilled artisan can determine an appropriate route of administration for a subject.

The present invention also provides, in certain embodiments, methods for treating a drug-resistant cancer (e.g., a cancer that includes cancer cells that have acquired resistance to one or more particular agents or drugs) in a subject (e.g., a subject in need thereof). As used herein, a cancer that is “drug-resistant” is a cancer that is not responsive to treatment with an agent (e.g., drug) that is administered using a non-targeted delivery method. The cancer may be resistant at the beginning of treatment, or it may become resistant during treatment. A drug-resistant cancer can be resistant to one or more different agents (e.g., drugs). In one embodiment, the drug-resistant cancer is resistant to treatment with a chemotherapeutic agent selected from doxorubicin (DOX), 6-mercaptopurine, 6-thioguanine, cytarabine, 5-fluorouracil decarbazine, mechlorethamine, thioepa chlorambucil, CC-1065, Melphalan (MEL), carmustine (BSNU), lomustine (CCNU), cyclothosphamide, busulfan, dibromomannitol, streptozotocin, mitomycin, cis-dichlorodiamine platinum (II) (DDP) cisplatin, daunorubicin, dactinomycin, bleomycin, mithramycin, anthramycin (AMC), vincristine, vinblastine, taxol, Paclitaxel (PTXL), maytansinoids, cytochalasin B, gramicidin D, ethidium bromide, emetine, etoposide, tenoposide, colchicin, dihydroxy anthracin dione, mitoxantrone, mithramycin, 1-dehydrotestosterone, glucocorticoids, procaine, tetracaine, lidocaine, propranolol, puromycin, and calicheamicin.

The “responsiveness” or “non-responsiveness” of a cancer to treatment can be evaluated by any known methods of measuring whether cancer or a symptom of cancer is slowed or diminished. Such methods are well known to a person of skill in the art (e.g., physician) and include, but not limited to direct observation and indirect evaluation, by evaluating subjective symptoms or objective physiological indicators and more.

Drug resistance in various cancers is multifactorial. Over-expression of the efflux pumps (p-glycoprotein (Pgp) or multi drug resistance 1(MDR1)) in the cell membranes, expression of anti-apoptotic mechanisms and enhanced faulty DNA repair mechanisms, all contribute to acquiring drug resistance by cancer cells. Pgp/MDR1 efflux pumps expressed on the surface of cancer cells are very effective in the efflux of chemotherapeutic drugs or other small molecules that are delivered on the surface or gain entry into cancer cells by diffusion. However, if the drugs or the chemotherapeutic drugs can be delivered deep in the cytoplasm of cancer cells in the pro-drug format which are then released as active drugs intracellularly, then the efflux of the chemotherapeutic agents by drug-resistant cancer cells will not be as efficient, and therefore, more of the chemotherapeutic drugs would remain intracellularly and achieve greater cytotoxicity. Therefore, some aspects of the current invention provide methods for overcoming drug resistance by administering a therapeutically effective amount of a bispecific targeting molecule and a composition comprising plurality of agent-polymer conjugates such that the agent in the agent-polymer conjugate is delivered deep into the cancer cell, thereby avoiding efflux from the cancer cell mediated by Pgp/MDR efflux pumps.

In a particular embodiment, the drug-resistant cancer is characterized by an increased expression of Pgp/MDR in the cancer cell. In another embodiment, the drug-resistant cancer is characterized by a lack of expression of Pgp/MDR in the cancer cell. Delivery of the agent in the agent-polymer conjugates through endocytosis (e.g., endocytic pathway) in the drug-resistant cancer cell, avoids the efflux of the agent mediated by cell surface efflux receptors. As used herein, endocytosis is a form of active transport in which a cell transports molecules (e.g., bispecific antibodies) into the cell by engulfing them into a separate compartment surrounded by cell membrane. The components of the endocytic path way and the fate of the molecules entering this pathway are well known to a person of skill in the art. In one embodiment, the invention provides a method of overcoming drug resistance by the administration of a composition comprising a plurality of agent-polymer conjugates to a subject pre-targeted with a bispecific targeting molecule such that the bispecific targeting molecule and the agent-polymer conjugates bound to the bispecific targeting molecule are endocytosed into the cancer cell.

In certain embodiments, a drug-resistant cancer is a cancer in which the cancer cells have acquired resistance to doxorubicin. In certain embodiments, a drug-resistant cancer is a cancer in which the cancer cells have acquired resistance to paclitaxel. In certain embodiments, a drug-resistant cancer is a cancer in which the cancer cells have acquired resistance to melphalan. In certain embodiments, a drug-resistant cancer is a cancer in which the cancer cells have acquired resistance to both doxorubicin and paclitaxel . In certain embodiments, a drug-resistant cancer is a cancer in which the cancer cells have acquired resistance to both paclitaxel and melphalan. In certain embodiments, a drug-resistant cancer is a cancer in which the cancer cells have acquired resistance to both doxorubicin and melphalan. In certain embodiments, a drug-resistant cancer is a cancer in which the cancer cells have acquired resistance to doxorubicin, paclitaxel and melphalan.

In other embodiments, the methods described herein are useful for treating cell proliferative disorders other than cancer including, but not limited to, adrenal cortex hyperplasia (Cushing's disease), congenital adrenal hyperplasia, endometrial hyperplasia, benign prostatic hyperplasia, breast hyperplasia, intimal hyperplasia, focal epithelial hyperplasia (Heck's disease), sebaceous hyperplasia, and compensatory liver hyperplasia.

### Compositions Comprising Agent-Polymer Conjugates of the Invention

The present invention also provides, in further embodiments, compositions comprising agent-polymer conjugates of the invention. In one embodiment, the compositions comprise a plurality of agent-polymer conjugates. The plurality of agent-polymer conjugates can include, for example, a population of multiple agent-polymer conjugates, a mixture of at least two different populations of single agent-polymer conjugates, wherein each population in the mixture comprises a different agent in comparison to other populations in the mixture, or a combination of multiple agent-polymer conjugates and single agent-polymer conjugates (e.g., in any ratio).

In certain embodiments, the polymeric carrier of the agent-polymer conjugate comprises a structure represented by at least one of formulae

A-(X)—Pₙ—(X), (III)

A-(X)—Pₙ—(Y), (IV)

A-(X)—Pₙ—(X)-A, (V)

A-(X)—Pₙ—(Y)-A, (VI)

wherein (X), P and (Y) are independently an amino acid with a non-polar side chain, an amino acid with a polar side chain that is not charged at physiological pH, or an amino acid with a polar side chain that is charged at physiological pH (e.g., glutamic acid, lysine); wherein the agent is covalently linked to (P); wherein n is at least one; and wherein A is a target moiety (e.g., diethylene triaminepentaacetic acid (DTPA) that is recognized by a binding site on a bispecific targeting molecule. In other embodiments, (X), P and (Y) are independently glucose or an amino-sugar (e.g., glucosamine, fructoseamine, galactosamine). In a particular embodiment, the agent-polymer conjugates comprise at least one chemotherapeutic agent (e.g., doxorubicin, paclitaxel or methotrexate). In one embodiment, the plurality of agent-polymer conjugates comprises a population of DTPA-Doxorubicin-Paclitaxel-Melphalan-Poly Glutamic acid (D-Dox-PTXL-MEL-PGA) conjugates. In another embodiment, the plurality of agent-polymer conjugates comprises a population of DTPA-Doxorubicin-Paclitaxel-Poly Glutamic acid (D-Dox-PTXL-PGA) conjugates. In another embodiment, the plurality of agent-polymer conjugates comprises a mixed population of DTPA-Doxorubicin-Poly Glutamic acid (D-Dox-PGA) conjugates and DTPA-Paclitaxel-Poly Glutamic acid (D-PTXL-PGA) conjugates (e.g., as shown in Examples 8-14)

In certain embodiments, the compositions comprising agent-polymer conjugates of the invention are pharmaceutical formulations comprising a plurality of agent-polymer conjugates, and one or more pharmaceutically-acceptable carriers or excipients. Such pharmaceutical formulations are suitable for use in treating cancer in a subject in need thereof (e.g., drug-resistant cancers).

The pharmaceutical formulations described herein typically comprise an effective amount (e.g., therapeutically effective amount) of an agent described herein and one or more pharmaceutically acceptable excipients, vehicles diluents, stabilizers, preservatives, solubilizers, emulsifiers, adjuvants and/or carriers. For example, such pharmaceutical compositions can include diluents of various buffer content (e.g., Tris-HCl, phosphate), pH and ionic strength; additives such as detergents and solubilizing agents (e.g., Polysorbate 20, Polysorbate 80), anti-oxidants (e.g., ascorbic acid, sodium metabisulfite), preservatives (e.g., Thimerosol, benzyl alcohol) and bulking substances (e.g., lactose, mannitol); see, e.g., Remington's Pharmaceutical Sciences, 18th Edition (1990, Mack Publishing Co., Easton, Pa.) pages 1435:1712, which are herein incorporated by reference.

Depending on the intended mode of administration, the pharmaceutical formulations can be in a solid, semi-solid, or liquid dosage form, such as, for example, tablets, suppositories, pills, capsules, microspheres, powders, liquids, suspensions, creams, ointments, lotions or the like, possibly contained within an artificial membrane, preferably in unit dosage form suitable for single administration of a precise dosage. Suitable doses per single administration of an agent include, e.g., doses of about or greater than about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 20 mg, about 30 mg, about 40 mg, about 50 mg, about 100 mg, about 200 mg, about 300 mg, about 400 mg, about 500 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, or about 3,000 mg. Each dose can be administered over a period of time deemed appropriate by a skilled practitioner.

In some embodiments, the pharmaceutical formulation further comprises one or more additional agents that are not covalently linked to the polymeric carrier of the agent-polymer conjugate. In certain embodiments, the additional agent is a therapeutic agent. In certain embodiments, the additional agent is a non-therapeutic agent. In a particular embodiment, the non-therapeutic agent is an agent used for diagnostic purposes (e.g., fluorescein or other labeling agent specific for cancer cells).

### Kits Comprising Agent-Polymer Conjugates of the Invention

In additional embodiments, the present invention provides kits that comprise at least one agent-polymer conjugate of the invention. Any of the agent-polymer conjugates described herein are suitable for inclusion in the kits. In a particular embodiment, the kits also include at least one bispecific targeting molecule.

Typically, the kit comprises a plurality of agent-polymer conjugates of the invention, wherein the plurality comprises either a population of multiple agent-polymer conjugates, each multiple agent-polymer conjugate comprising at least two different agents for inhibiting the growth or metastasis of a cancer cell covalently attached to a polymeric carrier, or a mixture of at least two different populations of single agent-polymer conjugates, each single-agent polymer conjugate comprising an agent for inhibiting the growth or metastasis of a cancer cell covalently linked to a polymeric carrier, wherein each population in the mixture comprises a different agent in comparison to other populations in the mixture; or a combination multiple agent-polymer conjugates and single agent-polymer conjugates.

In certain embodiments, the kit comprises agent-polymer conjugates comprising one or more chemotherapeutic agents (e.g., doxorubicin, paclitaxel, melphalan), in one or more containers.

In some embodiments, the kits further include one or more additional component(s), such as, for example, one or more pharmaceutically-acceptable carriers or excipient, one or more diagnostic or detection reagents (e.g., for detecting cancer cells in a subject), directions/instructions for administration, and relevant dosage information.

Typically, the kits are compartmentalized for ease of use and can include one or more containers with reagents. In one embodiment, all of the kit components are packaged together. Alternatively, one or more individual components of the kit can be provided in a separate package from the other kits components. In some embodiments, the other kit components can include instructions and/or illustrations that provide instructions for the use of components in the kit.

As used herein, the singular form “a” includes plural references unless the context clearly dictates otherwise. For example, the term “a population” may include a plurality of populations, including a mixed population containing multiple different group of molecules.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

### EXEMPLIFICATION

### Example 1

### Purification of Anti-Her2/Neu Affibodies

### Affibody Production

Affibodies were expressed as 6-His tag fusion proteins from pET28b vector encoding for affibody gene between NcoI and HINDIII restriction sites in E. coli strain BL21. 15 μl of bacteria was inoculated in 100 ml of Lucia-broth (LB) media containing 30 μg/ml of kanamycin in sterile 500 ml Erlenmeyer flask and incubated overnight at 370C shaker. 100 μl of the E. coli cells were taken from the overnight culture and inoculated to fresh LB media (300 ml) containing 30 μg/ml kanamycin and grown at 370 C. When A600 nm of 0.8 was achieved, gene expression was induced by the addition of 3 ml of isopropyl b-D-thiogalactoside (IPTG) at a final concentration of 1 mM. After allowing the cells to grow overnight at 370 C shaker, E. coli cells were harvested by ultracentrifugation (30,000 rpm, 30 mins, 40 C). The cells were then resuspended in 30 ml of binding buffer (50 mM Sodium phosphate, 0.3M NaCl, 5 mM Imidazole, 0.1% Triton X 100 1 mM PMSF, pH 8) and were lysed by 5 cycles of freeze thaw procedure using liquid nitrogen. After cell lysis, ultracentrifugation (30,000 rpm, 30 mins, 40 C) was carried out to separate the cell lysate from the cell debris.

### Affibody Purification:

The 6-His-Her2/neu fusion proteins were recovered using Profinity™ Immobilized metal affinity chromatography (IMAC) Ni2+-charged resin (Bio-Rad). 1 ml of the IMAC resin slurry was taken and the storage solution was removed using magnetic rack. IMAC column were then washed with 3 column volumes of distilled water and added enough distilled water make 50% slurry. 30 ml of the cell lysate containing 6-His-6-Her2/neu proteins was then added to the prepared resin slurry and swirled mixture gently. Resin-lysate mixture was then incubated at 40 C for 30 minutes and then mixture was loaded to the column. After the resin had settled down in the column, the cell lysate flow through the column was collected and the column was washed with 5 volumes of binding/washing buffer (50 mM Sodium Phosphate, 0.3M NaCl, 5 mM Imidazole, pH8). After thorough washing proteins were eluted using 5 ml of the elution buffer (50 mM Sodium phosphate, 0.3M NaCl, 0.5M Imidazole, pH8). Protein concentration was determined using Pierce Bicinchoninic acid assay (BCA) kit with bovine serum albumin (BSA) as the standard.

### Example 2

### Characterization of anti-Her2/Neu Affibodies

### SDS PAGE Identification of Anti-HER2/neu Affibody:

Bio-Rad mini-PROTEAN Tetra cell kit was used for the characterization of purified Affibody using SDS PAGE. Affibody molecule consists of a C-terminal cysteine residue with free sulfhydryl residue, which tends to oxidize and form dimers. Therefore, both the reduced (treated with 20% β mercaptoethanol) and non-reduced samples were analyzed in the same gel. Hand-cast gels were made with Acrylamide/Bis-acrylamide with 12.5% resolving gel and 4% stacking gel (around 2 cm). After polymerization of gel, 6 μg of protein samples in loading buffer containing 10% SDS and bromophenol blue tracking dye were prepared. Samples were heated at 95° C. for 10 minutes before loading samples to the gel. SDS-PAGE running buffer (25 mM Tris, 192 mM glycine, 0.1% SDS) was used for gel electrophoresis at 90V for about 95 minutes. After completion of electrophoresis, gel was removed from the gel cassette, rinsed with deionized water for 10 minutes and then stained with 0.1% coomassie brilliant blue for 30 minutes. Gels were then destained with three changes of the de-staining solution (50% deionized water, 40% methanol, 10% glacial acetic acid). The gels were then rinsed with deionized water and then transferred to wet chromatographic filter paper followed by overlaying with plastic sheet. The assembly was then transferred to Bio-Rad gel dryer (Model No. 583) for 2 hours under vacuum.

Anti-HER2/Neu Affibody Labeling with FITC:

The cysteine residue at the C-terminal residue of affibody was used for the site-specific labeling of affibody using thiol-reactive Fluorescein-maleimide dyes. 0.5 mg/ml of Affibody was incubated with 20 mmol/L of dithiothreitol (DTT) at pH 7.4 for 2 hours at room temperature. After the reduction of oxidized cysteine, affibody solution was dialyzed extensively against 0.1M PBS buffer containing 10 mM EDTA for 24 hours at 37° C. Fluorescein-maleimide dye were then dissolved in DMSO and then added to the reduced affibody and reaction was allowed to proceed overnight at 4° C. Unreacted dyes were then removed by Sephadex G-10 desalting column chromatography using spin protocol.

### Epi-Fluorescent Microscopy Studies for Characterization of HER2/Neu Receptors in SKOV3 and SKOV3 TR Cell Lines:

SKOV3 and SKOV3 TR (Paclitaxel resistant) cell lines were obtained from Dr. Torchilin's lab and were cultured in RPMI 1640 medium with 10% Fetal clone (Thermo Fisher, USA), penicillin (1000 units/ml) and streptomycin (1000 units/ml) at 37° C. with 5% CO₂. Around 500 μl of culture media containing 80,000 SKOV3 and SKOV3 TR cells were added to the 12 well culture plates coverslip and incubated overnight. Cells were then washed with 0.1M PBS, after which they were fixed and permeabilized by adding 500 μl of Acetone to the wells for 10 minutes at room temperature, following which they were blocked with 3% BSA for 2 hours and washed again.100 μl of 5 μg/ml of Affibody-FITC was added to each coverslips and incubated in dark for 1 hour in a humidifier chamber. Coverslips were washed 5× times with PBS-T followed by PBS and were counterstained with Hoechst, and mounted on the slide with one drop of Fluoromount-G (Southern Biotech). Slides were then sealed using clear nail polish and were stored in slide box at −20° C. for subsequent epifluorescence microscopic examination (Nikon Eclipse from Dr. Torchillin's lab).

Flow Cytometry Studies for Characterization of HER2/Neu Receptors in SKOV3 and SKOV3 TR Cell Lines:

Cultured SKOV3 and SKOV3 TR cells were cultured in 6 well plates starting with 40,000 cell/well. After 70-80% confluency, cells were trypsinized and neutralized with RPMI 1640 cell culture medium. Then, the cell pellets were suspended in 100 μl of 0.1M PBS. The cells were then treated with either 100 μl of either 5μg/ml Affibody-FITC or 1% BSA alone and incubated at 40 C for 30 minutes. The cells were then washed 3× with ice cold 0.1M PBS. Samples were then assessed by flow cytometry (FACS Calibur instrument, BD Biosciences, San Jose, Calif.) equipped with an argon-ion laser and an optional second red diode laser (source energy, 15 mW; detection time, 500 counts per second). Data were live gated for 10,000 cells each by Forward light scatter (F SC) and Side light scatter by FL1 (blue laser, 488 nm). Cell Quest pro software was used for data acquisition and analysis (BD Biosciences, San Jose, Calif.).

### Example 3

### Preparation and Characterization of Anti-HER2/Neu X Anti-DTPA Fab Bispecific Targeting Molecule

### Preparation of Anti-DTPA Fab:

Intact monoclonal antibody anti-DTPA (2C31E11C7) was subjected to enzymatic digestion with immobilized papain beads (Pierce) to prepare Fab fragments. 3 mg/ml of the intact anti-DTPA was dialyzed overnight against the sample buffer (20 mM sodium phosphate, 10 mM EDTA, and pH 7). Immobilized papain beads were then equilibrated in digestion buffer containing 20 mM Sodium phosphate, 10 mM EDTA, 20 mM cysteine hydrochloride pH 7 and then added to the dialyzed sample followed by incubation for 20 hours at 37° C. shaking water bath. After incubation crude digest containing Fab and Fc fragments were separated from the immobilized papain beads, and mixed with 1 ml of 1.5 M Tris-HCl pH 7.5. Crude digest was then dialyzed overnight against the binding buffer (20 mM Sodium phosphate, 0.15M NaCl, pH 8) for the Protein A affinity purification of Fab fragments from Fc and undigested intact anti-DTPA antibody. Dialyzed crude digest was then applied to the Protein-A column and the pure anti-DTPA Fab fragment was collected in the fall through whereas Fc and intact anti-DTPA bound to the column. Anti-DTPA Fab fragments were then characterized using SDS-PAGE and ELISA.

### Immunoreactivity ELISA for Anti-DTPA Fab:

To check the immunoreactivity of anti-DTPA Fab fragments 100 μl of DTPA-BSA (1 μg/ml ) in 0.1M PBS was coated in 96 well microtiter plate (BD Falcon) and incubated at 37° C. for 1 hour. Plate was washed 5× with 0.1M PBS-T and then blocked by adding 200 μl of 3% BSA for 1 hour at 37° C. After, blocking the plates were washed with 0.1M PBS-T (5×) and then 100 μl serial dilutions of anti-DTPA Fab fragments starting with 1 μg were loaded to the plate. Intact anti-DTPA antibody was used as the positive control and anti-myosin antibody as the negative control. Plates were then incubated for 1 hour at 37° C., following which they were washed with 0.1M PBS-T (5×). 50 μl/well of Secondary antibody Goat anti-Mouse antibody conjugated to HRP was added to the plate and incubated for 1 hr at 37° C. and washed with 0.1M PBS-T (5×). Finally 50 μl/well of K-Blue substrate was added to the plate and incubated in dark at room temperature for 15 minutes. Plate was then read at 630 nm and results were analyzed using GEN5.0 software (BioTek instruments).

### Preparation of Anti-HER2/Neu X Anti-DTPA Fab Bispecific Targeting Molecule:

Purified Anti-HER2/affibody was used for the generation of the bispecific complex. Anti-DTPA Fab fragment (1 mg/ml) in 0.1 M PBS pH 7.4 was modified with 100× molar excess of N-hydroxy succinimide ester of Bromoacetic acid and the reaction was allowed to proceed for 6 hr at 4° C. Modified anti-DTPA was then purified using Sephadex G-25 prepacked column (GE Healthsciences) using spin protocol. 0.1M PBS pH 7.4 was used as the elution buffer. The extent of modification of anti-DTPA was assessed using 2,4,6-Trinitrobenzene sulfonic acid assay and anti-DTPA ELISA was run to check the immunoreactivity of modified anti-DTPA as described in step 3.2. Dimeric anti-HER2/neu affibody were reduced with 20 mM DTT for 2 hours at room temperature following which they were dialyzed overnight against 4 liters of 0.1M PBS, 10 mM EDTA pH 7.4. Equi-molar concentration of bromoacetylated anti-DTPA and reduced affibody with free thiol groups were mixed together and incubated overnight at 4° C. This led to the conjugation between the two via thioether linkage.

### Purification of Bispecific Targeting Molecule:

Crude reaction mixture was passed through the Profinity™ IMAC column. Unreacted anti-DTPA Fab fragment didn't bind to the column and was obtained in the flow through. Bound multimeric and bispecific complex along with the free unconjugated affibody were eluted out from the column using 1 ml of the elution buffer (50 mM Sodium phosphate, 0.3M NaCl, 0.5M Imidazole, pH8). The eluent was then extensively dialyzed against 4L of 0.2 M phosphate buffer pH7.4 overnight using 20,000-kDa molecular weight cutoff membrane. HPLC size exclusion chromatography was then used for the separation of the bispecific complex from the multimeric complexes. For HPLC Zorbax GF-250 column (9.4×250 mm) (Agilent Technologies, size exclusion limits =400,000 Daltons to 10,000 Daltons) equilibrated with 0.2M phosphate buffer was used. 400 μl of the sample was applied to the column and 250 μl aliquot fractions were collected. Absorbance at 280 nm was read to determine the elution profile.

### Example 4

### Synthesis and Characterization of Polymer Linked to Target Moiety Conjugation of DTPA to PGA

A solution of 50 mg/ml of Poly Glutamic acid (PGA) in 0.1 M sodium bicarbonate pH 8.6 was prepared. 3× excess of diethylene triaminepentaacetic acid (DTPA) dissolved in minimum quantity of DMSO was added dropwise to PGA solution while vortexing it vigorously. The mixture was incubated at room temperature for 4 hours and then extensively dialyzed overnight at 4° C. in 4 liters of 0.1M phosphate buffered saline. Conjugation of DTPA to PGA was then analyzed using 2,4,6-Trinitrobenzene sulfonic acid assay. TNBS reacts with free amine groups to form a chromogenic derivative, which then can be quantitated by measuring absorbance at 420 nm. Unmodified PGA was used as the standard for comparison.

### Anti-DTPA ELISA for the Detection of PGA-DTPA:

A 96 well plate microtiter plate (BD Falcon) was coated with 100 μl of DTPA-BSA (1 μg/ml) in each of the 12 wells in row A and B of the plate. Row C and D are coated with 100 μl of DTPA-PGA (1 μg/ml) and incubated at 37° C. water bath for 2 hours. Plates were then washed 5× with 200 μl of 0.1M PBS containing 0.1% Tween 20 (PBST) pH7.4 and then 200 μl of 3% bovine serum albumin was added for blocking. After incubating the plate at 37° C. for 1 hour, plate was again washed as before and serial dilution of primary antibody 2C31E11C7 (10, 1, 0.1, 0.01, 0.001 μg/ml) was added in aliquots of 100 μl in quadruplicates (n=4). Plates were then incubated at 37° C. and washed with 0.1M PBST (pH 7.4). 50 μl aliquots of 1:500 dilution of secondary antibody Goat anti-mouse horseradish peroxidase (GAM-HRP) was then added to the wells and incubated at 37° C. Plates were washed with 0.1M PBST (pH7.4) and then 50 μl of substrate K-Blue is added to each wells. Plates are incubated at dark for 15 minutes at room temperature and plate is read at 630 nm using BioTek microplate reader. The results are then analyzed using GEN 5 software.

### Example 5

### Synthesis of Agent-Polymer Conjugates

### Conjugation of Doxorubicin to DTPA-PGA:

1 ml of 10 mg/ml of DTPA-PGA in 0.1M PBS pH 7.4 was mixed with 9.6 mg of doxorubicin (24 molar excess) dissolved in minimum amount of DMSO (300 μl). 17.2 mg of 1-ethyl-3-(3-dimethylaminopropyl) carbodiimide (EDC) was dissolved in minimal amount (300 μl) of DMSO and was added dropwise to the mixture of DTPA-PGA and doxorubicin while vortexing it vigorously. EDC leads to the activation of the carboxylic group in PGA, which then reacts, with the free amine group doxorubicin to form an amide bond. The reaction mixture was then incubated at 4° C. for 2 hours, followed by overnight incubation at room temperature at dark. Free unconjugated doxorubicin was then separated from the DTPA-doxorubicin-PGA conjugate by gel filtration chromatography using Sephadex G-25 columns (1×35 cm column). The cut off range of this column was 5000 Da with fractionation range of 1000-5000 Da. Blue dextran was first passed through the column to determine the void volume of the column and then the sample was added to column.1 ml (20 drops) fractions were collected using fraction collectors and absorbance at 490 nm was determined. The concentration of doxorubicin in DTPA-doxorubicin-PGA conjugate was then determined using the doxorubicin standard curve at 490nm.

### Conjugation of Melphalan to DTPA-PGA:

1 ml of 10 mg/ml of DTPA-PGA in 0.1M PBS pH 7.4 was mixed with 4.2 mg of melphalan dissolved in minimum amount of DMSO (300 μl). 17.2 mg of 1-ethyl-3-(3-dimethylaminopropyl)carbodiimide (EDC) was dissolved in minimal amount (300 μl) of DMSO and was added dropwise to the mixture of DTPA-PGA and melphalan while vortexing it vigorously. The reaction mixture was then incubated at 4° C. for 2 hours, followed by overnight incubation at room temperature at dark. Free unconjugated melphalan was then separated from the DTPA-melphalan-PGA conjugate by extensively dialyzing it against 4 liters 0.1M PBS pH 7.4 overnight at 4° C. The concentration of melphalan in DTPA-melphalan-PGA conjugate was then determined using the melphalan standard curve at 260 nm.

### Conjugation of Paclitaxel and DTPA to PGA:

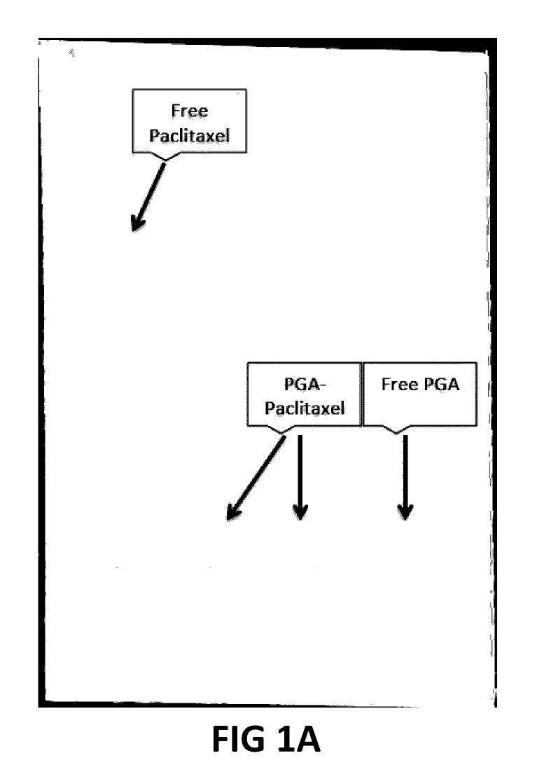

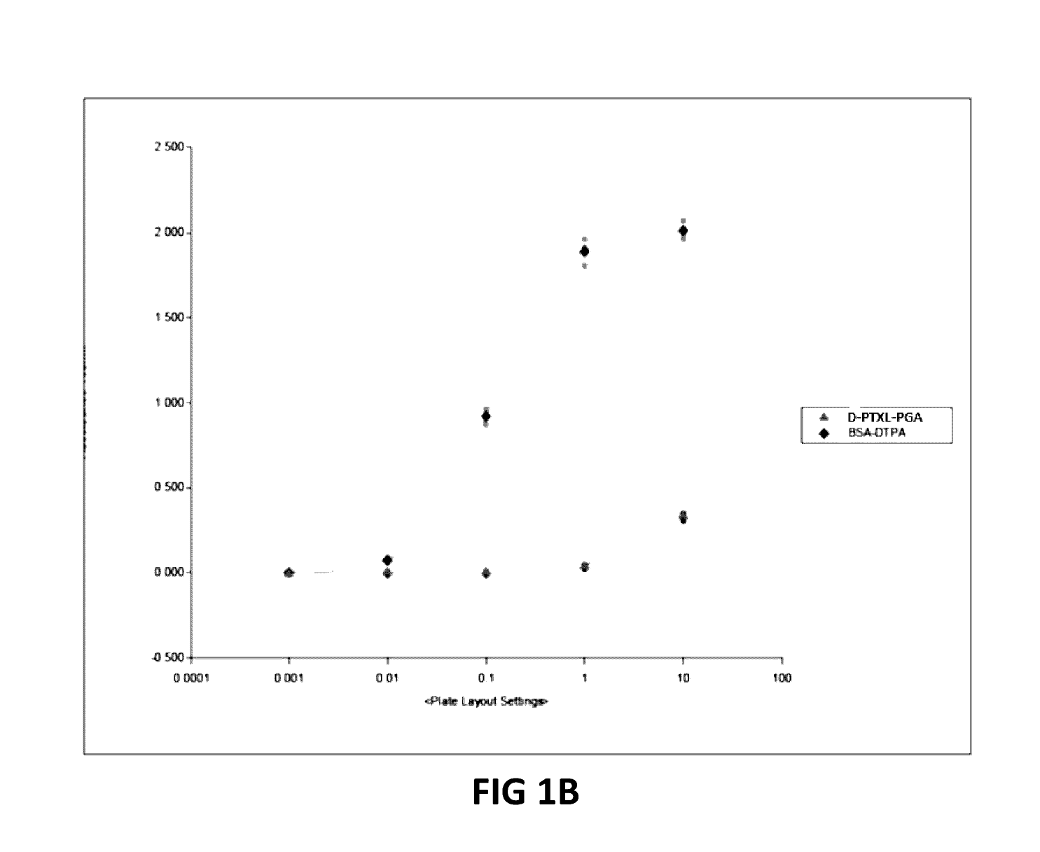

32 mg of PGA was dissolved in 1.5 ml of dry N,N-dimethylformamide. To this solution 11 mg of Paclitaxel, 15 mg of Dicyclohexylcarbodiimide, and a trace amount (3 mg) of dimethylaminopyridine was added. The reaction was allowed to proceed overnight at room temperature and Thin Layer Chromatography was performed to determine the conjugation of paclitaxel to the polymer. Reaction was stopped by pouring the reaction mixture in chloroform and polymer drug conjugate was then extracted using Rotavapor. The resulting precipitate was dissolved in 0.5 M sodium bicarbonate buffer (pH 9.6) and then dialyzed extensively overnight against 4 liters of 0.1 M sodium bicarbonate buffer (pH 9.6). 20× excess of DTPA was dissolved in minimum quantity of DMSO (200 μl) and was then added drop wise to the dialyzed Paclitaxel-PGA solution. The reaction mixture was incubate for 2 hours at room temperature and then dialyzed extensively against 0.1 M PBS pH 7.4 overnight at 4° C. Thin layer chromatography (TLC) analysis confirmed the conjugation of paclitaxel to PGA (FIG. 1A). An Anti-DTPA ELISA confirmed the conjugation of DTPA to the polymer and standard curve of Paclitaxel (227 nm) was plotted to determine the concentration of Paclitaxel in the DTPA-paclitaxel-PGA conjugate (FIG. 1B).

### Example 6

### Characterization of Agent-Polymer Conjugates

### Stability Studies of DTPA-Paclitaxel-PGA

The stability study of the DTPA-Paclitaxel-PGA was carried out in the various buffer systems at pH 4 and 7.4. An aliquot of 1 ml of DTPA-Paclitaxel-PGA solution was placed in the dialysis membrane bag with molecular cutoff of 3000 Da, closed with the clips, and placed in either into 50 ml of 0.1 M phosphate buffer solution media (pH 7.4) or 50 ml of 0.1 M sodium acetate buffer (pH4). The entire system was placed at 37° C. with continuous magnetic stirring. At various predetermined time intervals, 1 ml of samples were drawn from the release media and analyzed spectrophotometrically at 227 nm. Absorbance was taken 3 times for each sample and after which they were returned back to dialysate buffer.

### Measurement of Zeta Potential of Agent-Polymer Conjugates:

Zeta potential of the agent-Polymer conjugates were taken using Zeta Plus (zeta potential analyzer) Brookhaven Instruments Corporation (Holtsville, N.Y.) equipped with a palladium electrode with acrylic support was used. BIC zetapw32 software was used and all the measurements were taken at 25° C. using High Precision Mode. The zeta potential values for the various agent-polymer conjugates compared to the polymer (PGA) alone is shown in Table 1 below.

TABLE 1

| Agent-Polymer Conjugate | Zeta Potential at 25° C. (mV) |
|---|---|
| PGA | −21.425 |
| D-Dox-PGA | −11.475 |
| D-Paclitaxel-PGA | −15.754 |

### Example 7

### Binding Specificity of Bispecific Biotinylated Anti-DTPA to Biotin Receptors in Various Cell Lines

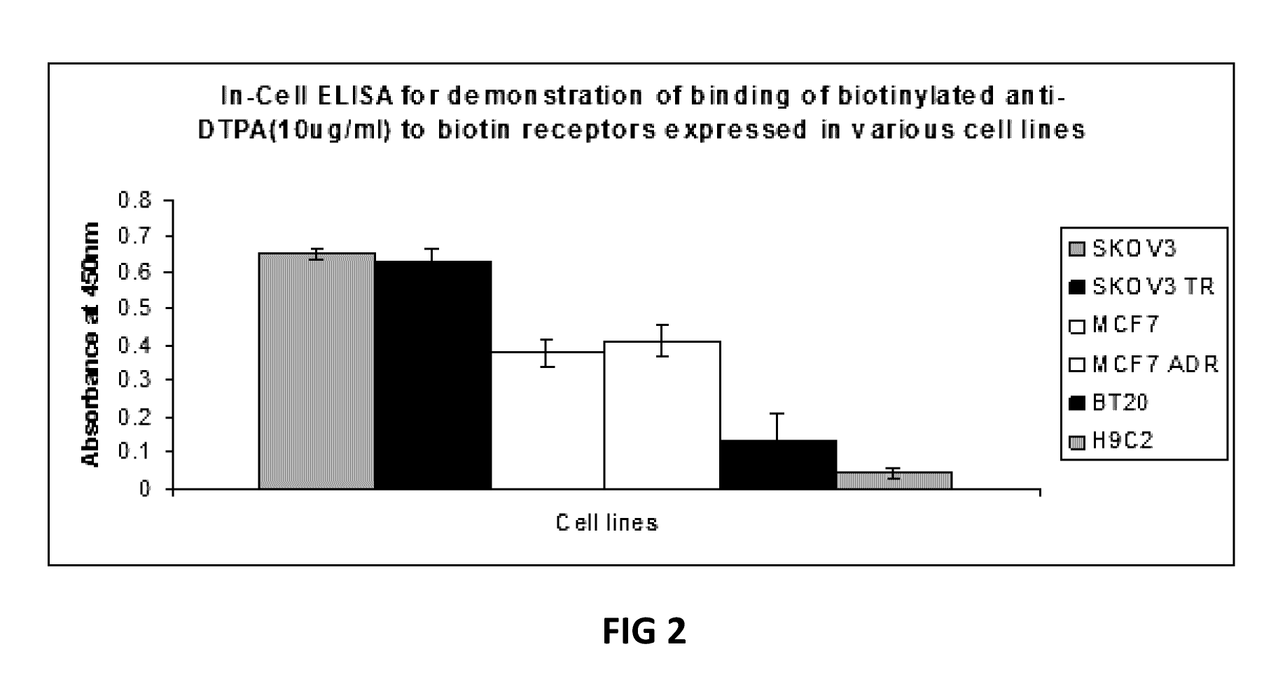

The bispecific biotinylated anti-DTPA antibody was prepared using standard procedures and methods exemplified herein (see, e.g., Examples 1 and 3). The standard procedures are well known to a person skilled in the art. FIG. 2 shows that bispecifc biotinylated anti-DTPA binds specifically to various cell lines that express biotin receptors on their surface.

### Example 8

### In Vitro Cell Viability Assay of Single or Mixed Agent-Polymer Conjugates in SKOV-3 Sensitive Ovarian Cancer Cells

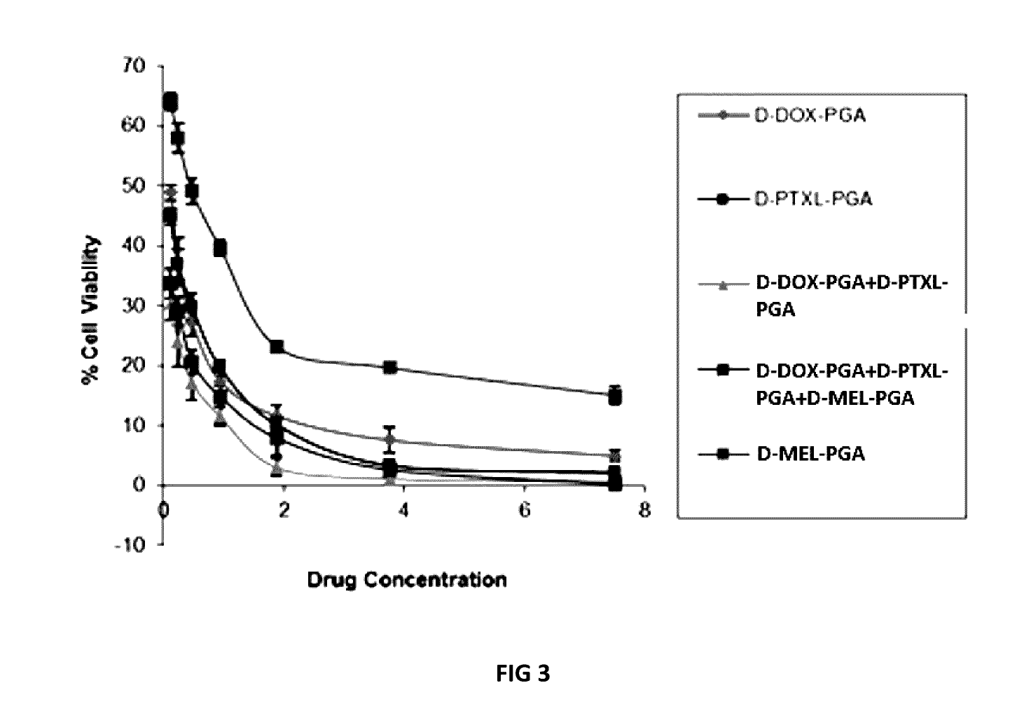

Human ovarian cancer (SKOV-3) sensitive cells were grown in six wellplates. Please provide description here. 5000 cells/well were seeded in the cell culture treated 96 well plates and were grown for 24 hours. Cultured cells (SKOV-3) were incubated with bispecific anti-Her-2 Affibody-anti-DTPA antibody for 24 hours at 37° C. After 24 h incubation, aliquots (1000 μl) of media containing serial concentrations of different agent-polymer conjugates Doxorubicin-DTPA-PGA (D-Dox-PGA), Paclitaxel-DTPA-PGA (D-PTXL-PGA) and DTPA-Melphalan-PGA (D-MEL-PGA) was added to the wells either as single agent-polymer conjugate or combinations of 2 or 3 single agent-polymer conjugate described above . After 24 h incubation at 37° C., viability was assessed by Trypan Blue exclusion test using CellTiter Blue® (Promega, Madison, Wis.) following the manufacturer's protocol. Briefly, media was removed from plates containing cells incubated with agent-polymer conjugates. The plate was washed 2× with 200 μl of complete medium and then incubated with 50 μl of 1:50 dilution of CellTiter Blue® reagent for 2 hours. Cell viability was then evaluated by measuring the fluorescence (excitation 530 nm, emission 590 nm) using a Synergy HT multi-21 detection microplate reader (Biotek, Winooski, Vt.). Cells treated with complete media alone were used as controls to calculate the 100% cell viability and the studies were carried out in triplicates in at least 3 different assays. FIG. 3 shows that all of the wells tested exhibited cytotoxicity to SKOV-3 sensitive cells. Wells with combinations of 2 or 3 single agent-polymer conjugates described above exhibited higher cytotoxicity relative to cytotoxicity exhibited by each of the corresponding single agent-polymer conjugate alone.

### Example 9

### In Vitro Cell Viability Assay of Single or Mixed Agent-Polymer Conjugates in SKOV-3 TR Resistant Ovarian Cancer Cells

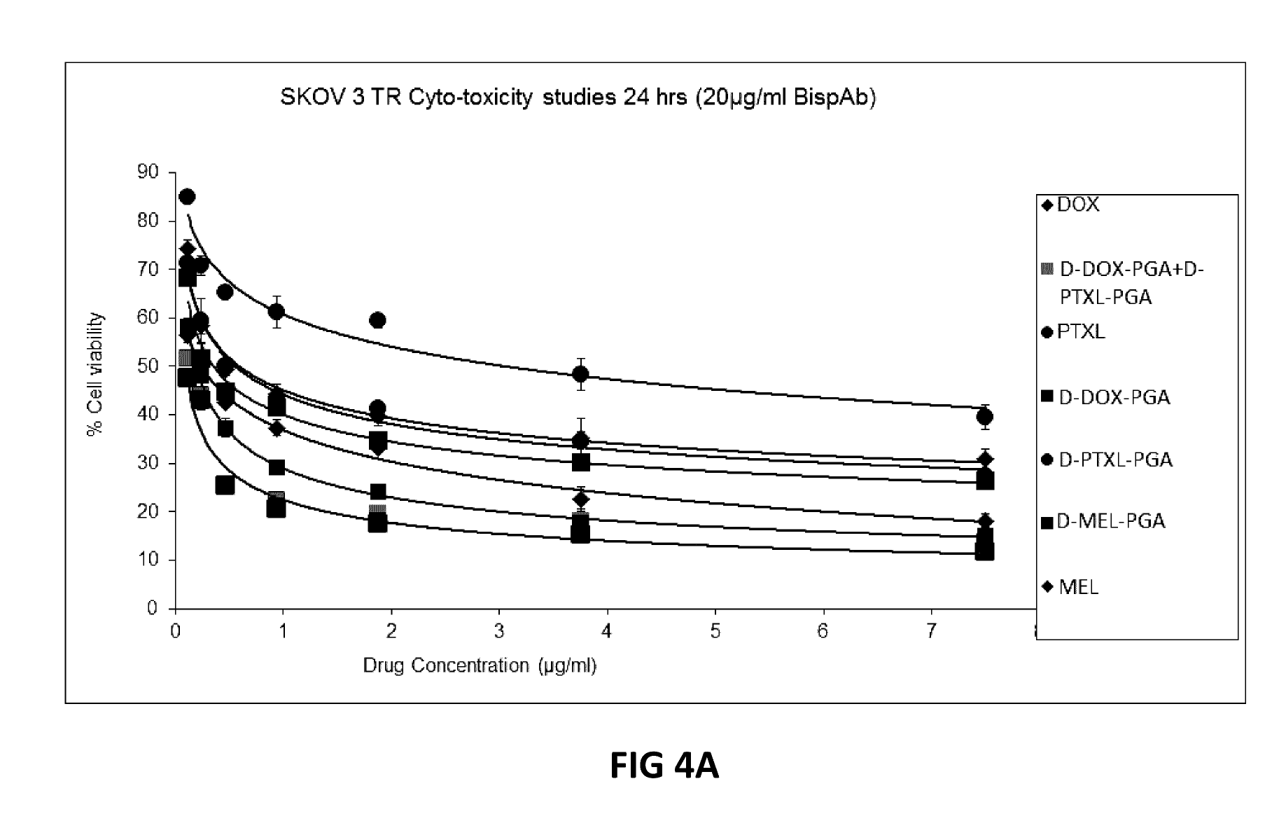

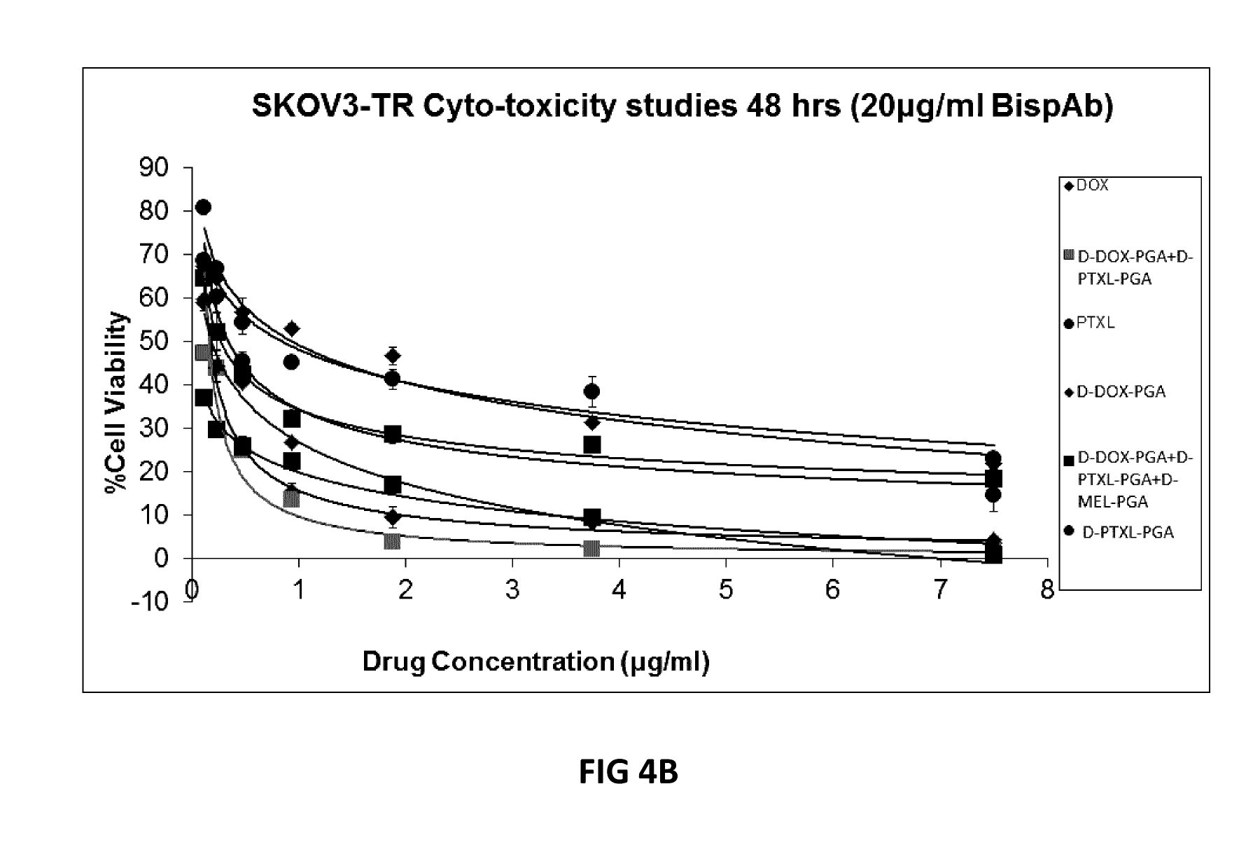

Human ovarian cancer (SKOV-3 TR) resistant cells were cultured using the same protocol described for culturing SKOV-3 TR resistant cells in Example 8 above. Cultured cells (SKOV-3 TR) were incubated with bispecific anti-Her-2 Affibody-anti-DTPA antibody at a concentration of 20 μg/ml for 24 hours at 37° C. After 24 h incubation, aliquots (1000 μl) of media containing serial concentrations of free agent (DOX, PTXL or MEL) or agent-polymer conjugates (Doxorubicin-DTPA-PGA (D-Dox-PGA), Paclitaxel-DTPA-PGA (D-PTXL-PGA) and Melphalan-DTPA-PGA (D-MEL-PGA)) was added to the wells either as single agent-polymer conjugate or combinations of 2 or 3 single agent-polymer conjugate described above. After 24-48 h incubation at 37° C., viability was assessed by Trypan Blue exclusion test using CellTiter Blue® (Promega, Madison, Wis.) following the manufacturer's protocol as described above in Example 8. FIGS. 4A and 4B show that all of the wells with any one of the single agent-polymer conjugate exhibited greater cytotoxicity to SKOV-3 TR resistant cells than the corresponding free agent. Wells with combinations of 2 or 3 single agent-polymer conjugate described above exhibited higher cytotoxicity relative to cytotoxicity exhibited by each of the corresponding single agent-polymer conjugate or the free agent. Each of the wells with combinations of 2 or 3 single agent-polymer conjugate described above showed similar therapeutic efficacy as shown in FIGS. 4A and 4B. In general, greater toxicity was observed after 48 hours incubation as compared to 24 hour incubation period.

### Example 10

### In Vitro Cell Viability Assay of Single or Mixed Agent-Polymer Conjugates in SKOV-3 TR resistant Ovarian Cancer Cells Pretargeted with 40 μg/ml of Bispecific Biotinylated-Anti-DTPA antibody.

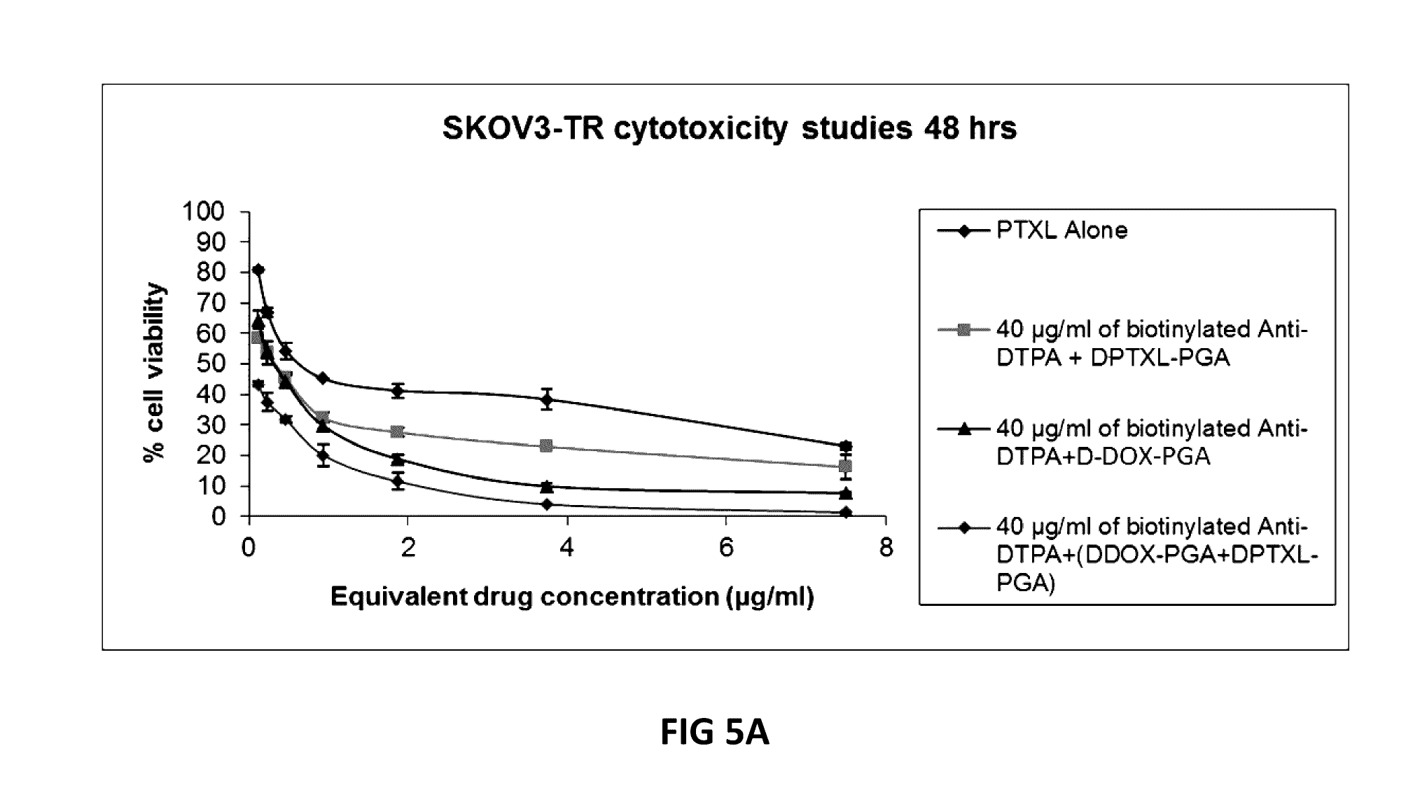

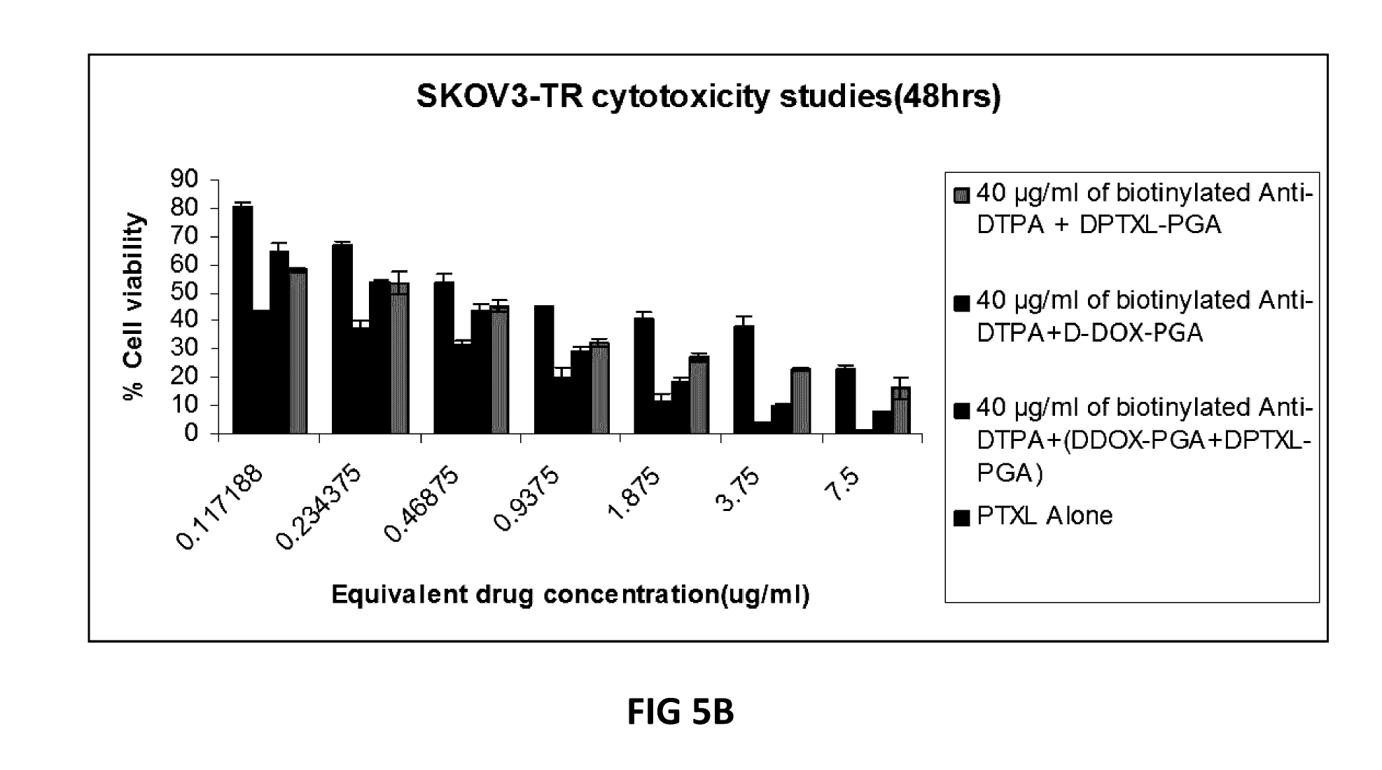

Human ovarian cancer (SKOV-3 TR) resistant cells were cultured using the same protocol described for culturing SKOV-3 TR resistant cells in Example 8 above. Cultured cells (SKOV-3 TR) were incubated with 40 μg/ml of bispecific biotinylated-anti-DTPA antibody for 24 hours at 37° C. After 24 h incubation, aliquots (1000 μl) of media containing serial concentrations of free agent (DOX or PTXL) or single agent-polymer conjugate (Doxorubicin-DTPA-PGA (D-Dox-PGA) or Paclitaxel-DTPA-PGA (D-PTXL-PGA) was added to the wells either as single agent-polymer conjugate or combinations of both single agent-polymer conjugate simultaneously. After 48 h incubation at 37° C., viability was assessed by Trypan Blue exclusion test using CellTiter Blue® (Promega, Madison, Wis.) following the manufacturer's protocol as described above in Example 8. FIGS. 5A and 5B show that all of the wells with any one of the single agent-polymer conjugate exhibited greater cytotoxicity to SKOV-3 TR resistant cells than the well with the corresponding free agent. Wells with combinations of both single agent-polymer conjugate described above exhibited higher cytotoxicity relative to cytotoxicity exhibited by each of the corresponding single agent-polymer conjugate or the free agent. FIG. 5B shows that the therapeutic efficacy increases with the concentration of the agent in all of the cases tested above. In general, greater toxicity was observed with higher concentrations in all the tested cases in this experiment. The therapeutic efficacy of the combination of two single agent-polymer conjugate described above is the highest at the highest effective concentration (8 μg/ml) tested here. The efficacy of the combination at an effective concentration of 8 μg/ml is much better than either of the single agent-polymer conjugate incubated separately, each at effective concentration of 8 μg/ml. Thus a much higher dose with greater efficacy can be reached with the combination of two or more single agent-polymer conjugates than each of the single agent-polymer conjugates incubated separately.

### Example 11

### In Vitro Cell Viability Assay of Single Agent-Polymer Conjugates in MCF-7 MDR Doxorubicin Resistant Mammary Carcinoma Cells

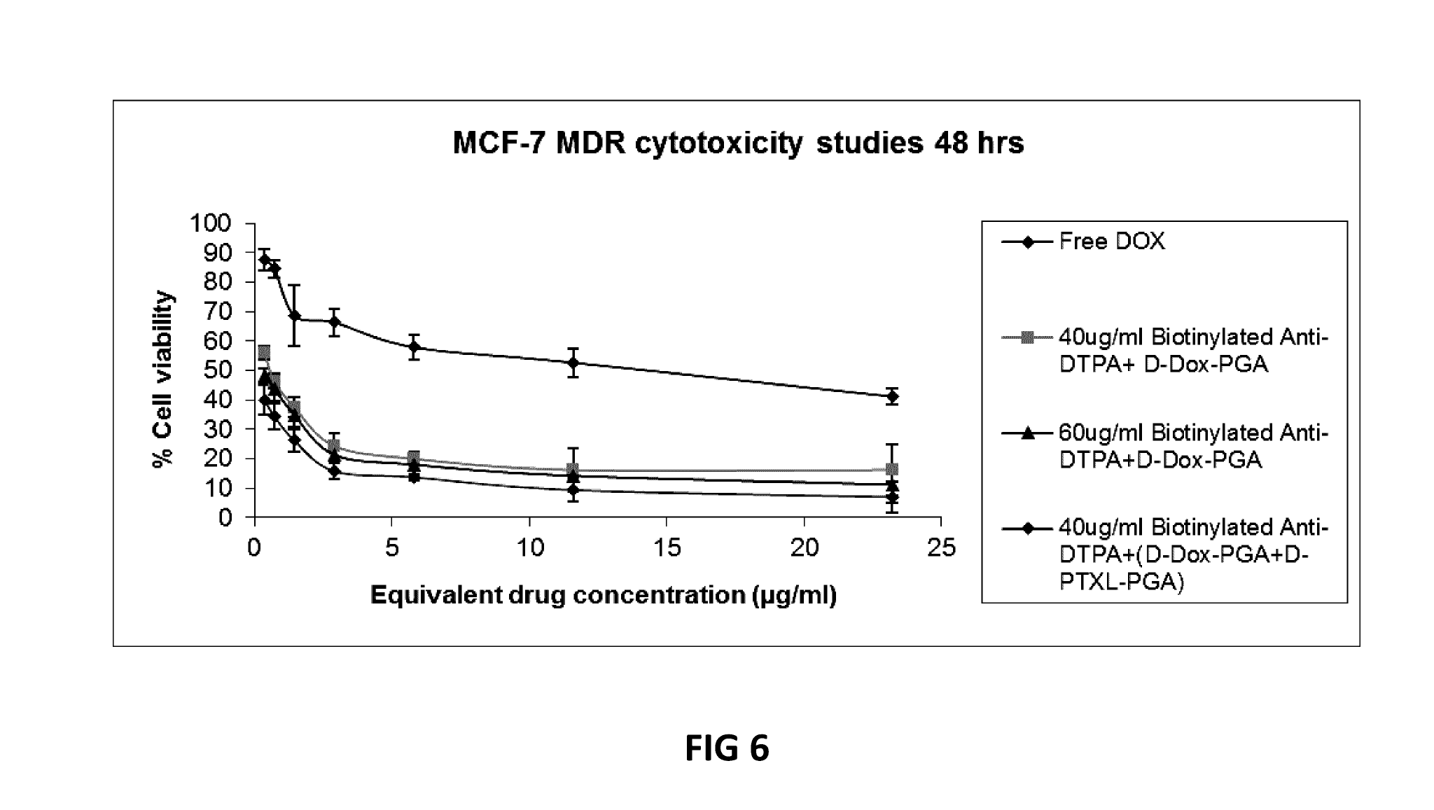

Human mammary carcinoma (MCF-7 MDR) Doxorubicin resistant cells were cultured using the same protocol described for culturing SKOV-3 sensitive cells in Example 8 above. Cultured cells (MCF-7 MDR) were incubated with 40 μg/ml of bispecific biotinylated-anti-DTPA antibody for 24 hours at 37° C. After 24 h incubation, aliquots (1000 μl) of media containing serial concentrations of free agent (DOX or PTXL) or single agent-polymer conjugate (Doxorubicin-DTPA-PGA (D-Dox-PGA) or Paclitaxel-DTPA-PGA (D-PTXL-PGA) were added to the wells either as single agent-polymer conjugate or combinations of both single agent-polymer conjugate simultaneously. After 48 h incubation at 37° C., viability was assessed by Trypan Blue exclusion test using CellTiter Blue® (Promega, Madison, Wis.) following the manufacturer's protocol as described above in Example 8. FIG. 6 shows that all of the wells with any one of the single agent-polymer conjugate exhibited greater cytotoxicity to MCF-7 MDR Doxorubicin resistant cells than the corresponding free agent. Wells with combinations of both single agent-polymer conjugate described above exhibited higher cytotoxicity relative to cytotoxicity exhibited by each of the corresponding single agent-polymer conjugate or the free agent.

### Example 12

### Determination of IC₅₀ Values of Paclitaxel or Paclitaxel-DTPA-PGA (D-PTXL-PGA) in SKOV-3 Sensitive and SKOV-3 TR Resistant Ovarian Cancer Cells

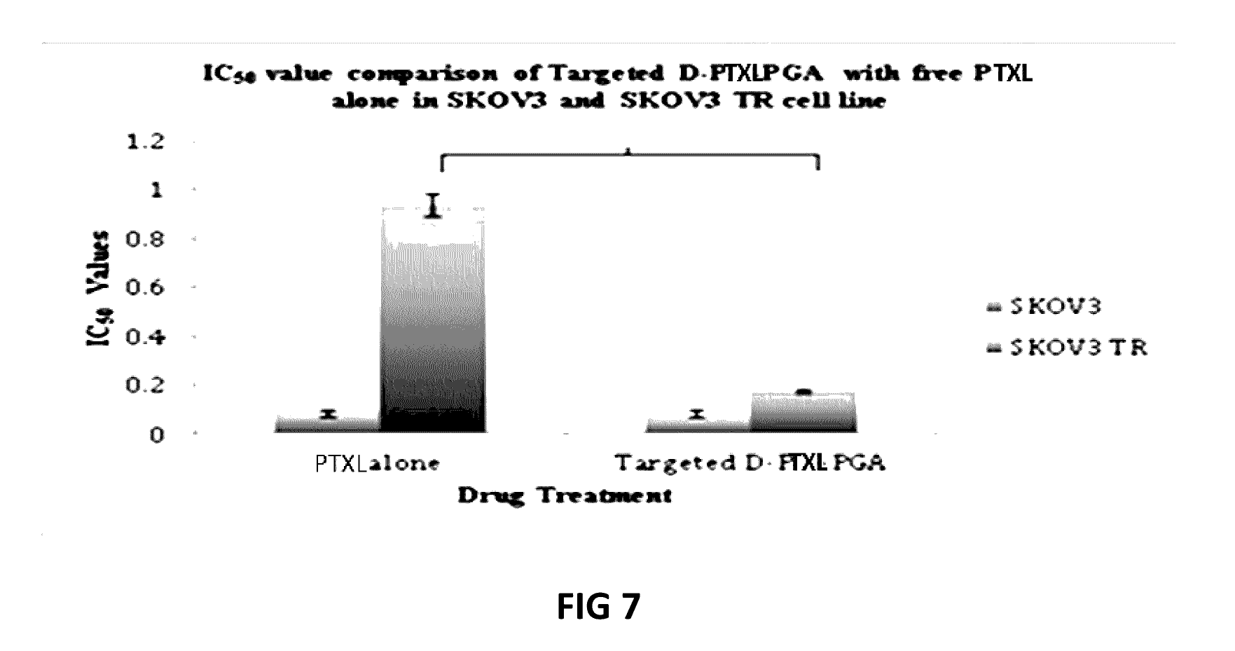

SKOV-3 sensitive and SKOV-3 TR resistant Ovarian cancer cells were cultured using the same protocol described in Examples 8 and 9 above. Cultured cells were incubated with bispecific anti-Her-2 Affibody-anti-DTPA antibody. After incubation, aliquots (1000 μl) of media containing free agent (PTXL) or single agent-polymer conjugate Paclitaxel-DTPA-PGA (D-PTXL-PGA) was added. IC₅₀value of free PTXL in SKOV-3 TR resistant cells (0.936 μg/ml) was about 10 times higher than the IC₅₀ value (0.172 μg/ml) of the corresponding species in SKOV-3 sensitive cells (FIG. 7). It was found that more of the free PTXL was retained in the SKOV-3 sensitive cells than in the SKOV-3 TR resistant cells. Also, it was found that same concentration of free PTXL exerted a greater degree of cytotoxicity in the sensitive cells than in the resistant cells. However, the IC₅₀ values of D-PTXL-PGA in paclitaxel sensitive (0.089 μg/ml) and resistant (0.069 μg/ml) ovarian cancer cells pretargeted with anti-HER2/neu affibody were comparable (FIG. 7). Unlike Free PTXL, it was found that D-PTXL-PGA was retained to the same extent or levels in the SKOV-3 sensitive and SKOV-3 TR resistant cells. These observed data demonstrate that Paclitaxel delivered as D-PTXL-PGA agent-polymer conjugate exhibited a higher cytotoxic effect and enhanced cell killing relative to Paclitaxel delivered as free drug on Paclitaxel resistant SKOV-3 TR Ovarian cancer cells.

### Example 13

### Delivery of Agent-Polymer Conjugates to MCF7-Doxorubicin Resistant Cells

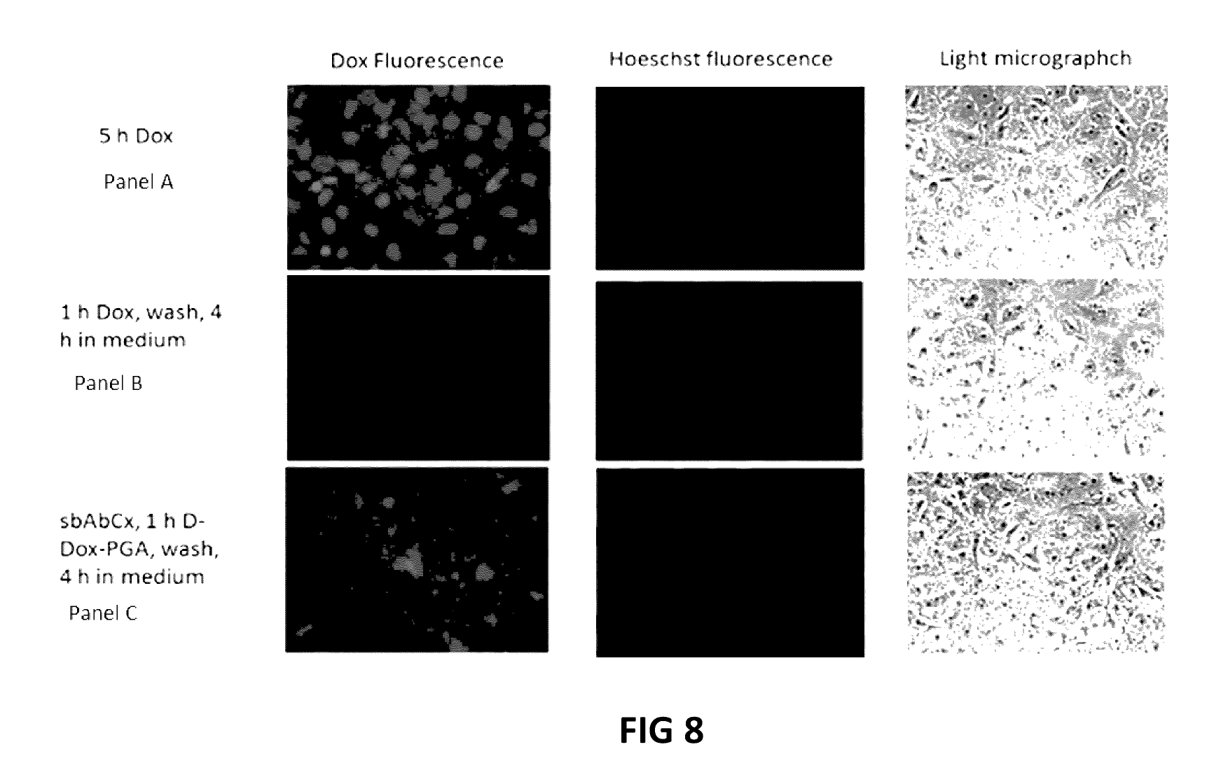

Human mammary carcinoma (MCF-7 MDR) Doxorubicin resistant cells were grown in six well plates. Cultured cells (MCF-7 MDR) were incubated with either bispecific biotinylated-anti-DTPA antibody (sbAbCx) or culture media alone for 30 min at 4° C. The cells were then washed 3× with cold 0.1 M PBS, and cells were incubated with either D-Dox-PGA or free Dox (15 μg/ml) at 37° C. for 1-5 h. Cells incubated with D-Dox-PGA and a batch of cells incubated with free Dox (15 μg/ml) were washed again with 3× with cold 0.1 M PBS, and they were incubated in fresh Dox free media for 4 h. Fluorescent intensity of treated cells was measured by obtaining digital fluoromicrographs of doxorubicin fluorescence in the samples using Olympus DP70 and X-cite 120. Fluorescence illumination system (excitation wavelength of 490 nm and emission wavelength of 520 nm). Fluorescent intensity data were analyzed using Image J software from NIH. All images were acquired at 245 ms exposure (FIG. 8). MCF-7 MDR cells incubated with 15 μg/ml free Dox for 5 h showed nuclear sequestration (FIG. 8, top row, Panel A) due to continuous presence of Dox. In cells incubated with free Dox for 1 h and in Dox free medium for 4 h, less Dox uptake occurred (FIG. 8, middle row, Panel B). However, when the cells are pretargeted with bispecific biotinylated-anti-DTPA antibody and then incubated with D-Dox-PGA, more Dox is retained in these cells (FIG. 8, bottom row, Panel C). These observed data demonstrate that Doxorubicin delivered as D-DOX-PGA agent-polymer conjugate are retained to a greater extent relative to free Doxorubicin delivered to human mammary carcinoma Doxorubicin resistant cells.

### Example 14

### Reduced Cardiocytotoxicity of Agent-Polymer Conjugates in H9C2 Rat Cardiomyocytes

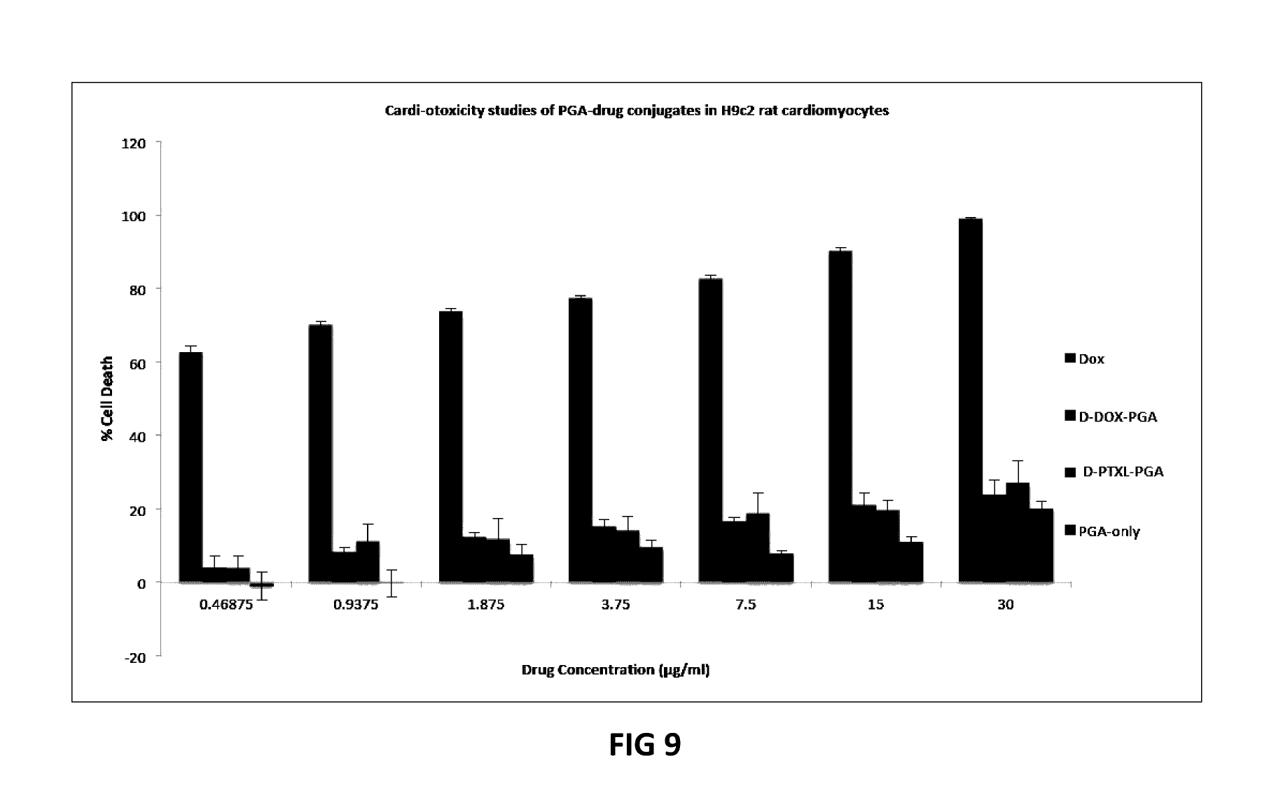

Rat embryonic cardiocyte (H9C2) purchased from American Type Culture Collection (VA, USA), was cultured in Dulbecco Minimum Essential Medium (Cassion Labs, Utah, USA) with 10% fetal clone (Thermo Fisher, USA), penicillin (100 U/ml), streptomycin (100 μg/ml), and amphotericin (0.25 μg/ml) at 37° C. in an atmosphere of 95% air and 5% CO₂. H9C2 cells (1×105 cells/well) were plated in six well plates, and at ˜80% confluence, they were used to assess cardiotoxicity. Quadruplicate cultures were treated with 3 ml of serial concentrations of free Dox or D-Dox-PGA or PGA alone or D-PTXL-PGA. Viability was assessed by Trypan Blue exclusion test using CellTiter Blue® (Promega, Madison, Wis.) following the manufacturer's protocol as described above in Example 8. FIG. 9 shows the toxicity (measured as % cell viability) of free Dox, D-Dox-PGA, PGA alone and D-PTXL-PGA relative to concentration of the drug. Irrespective of the concentration of free drug or the agent-polymer conjugate used, cardiocyte toxicity was significantly greater in free drugs(distinct fill pattern in the bar chart for free drugs) than in D-Dox-PGA (distinct fill pattern in the bar chart) and D-PTXL-PGA (distinct fill pattern in the bar chart). Even at the highest concentration (30 μg/ml) tested (FIG. 9), cardiocyte toxicity was lower in the cells incubated with agent-polymer conjugates (D-Dox-PGA or D-PTXL-PGA) compared to cells incubated with free drug (Dox or PTXL). Thus the data demonstrated that cardiocyte toxicity of the free drug was significantly reduced by using the various agent-polymer conjugates.

What is claimed is:

1. A method for inhibiting the growth or metastasis of a cancer cell, the method comprising:
a) contacting a cancer cell with a bispecific targeting molecule, wherein the bispecific targeting molecule comprises at least one first binding site for a target antigen on the surface of the cancer cell and at least one second binding site for a target moiety on an agent-polymer conjugate under conditions in which the bispecific targeting molecule binds to the cancer cell, thereby producing a cancer cell that is bound to the bispecific targeting molecule; and
b) contacting the cancer cell that is bound to the bispecific targeting molecule with a plurality of agent-polymer conjugates under conditions in which the bispecific targeting molecule that is bound to the cancer cell also binds to a target moiety on at least one agent-polymer conjugate, wherein the plurality of agent-polymer conjugates comprises:
i. a population of multiple agent-polymer conjugates, each multiple agent-polymer conjugate comprising at least two different agents for inhibiting the growth or metastasis of a cancer cell covalently linked to a polymeric carrier;
ii. a mixture of at least two different populations of single agent-polymer conjugates, each single-agent polymer conjugate comprising an agent for inhibiting the growth or metastasis of a cancer cell covalently linked to a polymeric carrier, wherein each population in the mixture comprises a different agent in comparison to other populations in the mixture; or
iii. a combination thereof,
thereby delivering the agent for inhibiting the growth or metastasis of a cancer cell to the cancer cell.

2. The method of claim 1, wherein the target antigen is a receptor or a ligand for a receptor.

3. The method of claim 1, wherein the polymeric carrier is uncharged or negatively charged at physiological pH.

4. The method of claim 3, wherein the polymeric carrier comprises at least three monomers.

5. The method of claim 4, wherein the monomers comprise organic molecules.

6. The method of claim 5, wherein the organic molecules are amino acids covalently linked by a peptide bond, poly-(D)-glucosamine, polyglycolic co-polymers or polyacetic acid copolymers.

7. The method of claim 6, wherein the polymeric carrier comprises a structure set forth in formulae I or II:
(X)—Rₙ—(X), (I)
(X)—Rₙ—(Y), (II)
wherein (X), R and (Y) are independently an amino acid with a non-polar side chain, an amino acid with a polar side chain that is not charged at physiological pH, or an amino acid with a polar side chain that is negatively charged at physiological pH; wherein the agent is covalently linked to (R); and wherein n is at least one.

8. The method of claim 7, wherein (X), R and (Y) are independently an amino acid with a polar side chain that is negatively charged at physiological pH.

9. The method of claim 8, wherein (X), R and (Y) are independently a glutamic acid residue or a lysine residue.

10. The method of claim 1, wherein the agent-polymer conjugates comprise an agent that is selected from the group consisting of a chemotherapeutic agent, a radioisotope, a cytokine, a pro-apoptotic agent, and an immune-activating agent.

11. The method of claim 10, wherein the agent is in a prodrug form.

12. The method of claim 10, wherein the agent is a chemotherapeutic agent.

13. The method of claim 12, wherein the chemotherapeutic agent is doxorubicin, paclitaxel or methotrexate.

14. The method of claim 1, wherein the target moiety is selected from the group consisting of diethylene triaminepentaacetic acid (DTPA), and dinitrophenol (DNP).

15. The method of claim 14, wherein the target moiety is diethylene triaminepentaacetic acid (DTPA).

16. The method of claim 1, wherein the bispecific targeting molecule comprises at least one antibody or antigen-binding fragment thereof.

17. The method of claim 16, wherein the antigen-binding fragment is an affibody.

18. The method of claim 1, wherein the covalent linkage is a peptide linkage, an amide linkage, a sulfyhydrl linkage, a thioester linkage, an ether linkage, an ester linkage, a hydrazine linkage, a hydrazine linkage, an oxime linkage or combinations thereof.

19. A method of treating a cancer in a subject in need thereof, the method comprising:
a) administering to a subject a bispecific targeting molecule, wherein the bispecific targeting molecule comprises at least one first binding site for a target antigen on the surface of a cancer cell in the subject and at least one second binding site for a target moiety on an agent-polymer conjugate; and
b) administering to the subject an effective amount of a composition comprising a plurality of agent-polymer conjugates, wherein the plurality of agent-polymer conjugates comprises:
i. a population of multiple agent-polymer conjugates, each multiple agent-polymer conjugate comprising at least two different agents for inhibiting the growth or metastasis of a cancer cell covalently linked to a polymeric carrier;
ii. a mixture of at least two different populations of single agent-polymer conjugates, each single-agent polymer conjugate comprising an agent for inhibiting the growth or metastasis of a cancer cell covalently linked to a polymeric carrier, wherein each population in the mixture comprises a different agent in comparison to other populations in the mixture; or
iii. a combination thereof,
thereby treating cancer in the subject.

20. The method of claim 19, wherein the subject is a mammal.

21. The method of claim 20, wherein the subject is a human.

22. The method of claim 19, wherein the bispecific targeting molecule is administered to the subject prior to administration of the composition comprising agent-polymer conjugates.

23. The method of claim 22, wherein the bispecific targeting molecule is administered to the subject at least about 1 to about 3 hours prior to administration of the composition comprising agent-polymer conjugates.

24. The method of claim 19, wherein the bispecific targeting molecule and the composition comprising agent-polymer conjugates are administered intravenously.

25. The method of claim 19, wherein the subject has a solid tumor.

26. The method of claim 19, wherein the subject has a hematological cancer.

27. The method of claim 19, wherein the cancer is a drug-resistant cancer.

28. The method of claim 27, wherein the drug-resistant cancer is a drug-resistant ovarian cancer or a drug-resistant breast cancer.

29. A composition comprising a plurality of agent-polymer conjugates, wherein the plurality comprises:
i. a population of multiple agent-polymer conjugates, each multiple agent-polymer conjugate comprising at least two different agents for inhibiting the growth or metastasis of a cancer cell covalently attached to a polymeric carrier;
ii. a mixture of at least two different populations of single agent-polymer conjugates, each single-agent polymer conjugate comprising an agent for inhibiting the growth or metastasis of a cancer cell covalently linked to a polymeric carrier, wherein each population in the mixture comprises a different agent in comparison to other populations in the mixture; or
iii. a combination thereof.

30. The composition of claim 29, wherein the polymeric carrier comprises a structure represented by at least one of formulae
A-(X)—Rₙ—(X), (III)
A-(X)—Rₙ—(Y), (IV)
A-(X)—Rₙ—(X)-A, (V)
A-(X)—Rₙ—(Y)-A, (VI)
wherein (X), R and (Y) are independently an amino acid with a non-polar side chain, an amino acid with a polar side chain that is not charged at physiological pH, an amino acid with a polar side chain that is negatively charged at physiological pH, an amino sugar, or glucose; wherein the agent is covalently linked to (R); n is at least one; and A is a target moiety.

31. The method of claim 29, wherein the agent is a chemotherapeutic agent.

32. The method of claim 31, wherein the chemotherapeutic agent is doxorubicin, paclitaxel or methotrexate.

33. The composition of claim 29, wherein (X), R and (Y) are independently a glutamic acid residue, a lysine residue or a polysaccharide.

34. The composition of claim 29, wherein A is DTPA or DNP.

35. A kit comprising:
a) the composition of claim 29; and
b) a bispecific targeting molecule comprising at least one first binding site for a target antigen on the surface of a cancer cell and at least one second binding site for the target moiety on the polymeric carrier.
